SEQUENCE LISTING (35 sequences)

SEQ ID NO: 1 - protein, 237 aa, synthetic construct
  VARIANT 45: note = X is any amino acid except K
  VARIANT 49: note = X is any amino acid except R
  VARIANT 73: note = X is any amino acid except Q
  VARIANT 76: note = X is any amino acid except L
  VARIANT 78: note = X is any amino acid except D
  VARIANT 82: note = X is any amino acid except M
  VARIANT 83: note = X is any amino acid except I
  VARIANT 84: note = X is any amino acid except I
  VARIANT 89: note = X is any amino acid except K
  VARIANT 102: note = X is any amino acid except E
  VARIANT 150: note = X is any amino acid except Q
  VARIANT 155: note = X is any amino acid except L
  VARIANT 181: note = X is any amino acid except S
  VARIANT 184: note = X is any amino acid except K
  VARIANT 193: note = X is any amino acid except Q
  VARIANT 206: note = X is any amino acid except A
  VARIANT 223: note = X is any amino acid except E
GESLFKGPRDYNPISSTICHLTNESDGHTTSLYGIGFGPFIITNXHLFXRNNGTLLVQSLHGVFKVKNTTTLXQHXIXGRDXXXIRMPXDFPPFPQKLKFRXPQREERICLVTTNFQTKSMSSMVSDTSCTFPSSDGIFWKHWIQTKDGXCGSPXVSTRDGFIVGIHSASNFTNTNNYFTXVPXNFMELLTNXEAQQWVSGWRLNXDSVLWGGHKVFMSKPEXPFQPVKEATQLMNE

SEQ ID NO: 2 - protein, 237 aa, synthetic construct
GESLFKGPRDYNPISSTICHLTNESDGHTTSLYGIGFGPFIITNKHLFFRNNGTLVVQSLHGVFKVKNTTTLQQHPIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 3 - protein, 237 aa, synthetic construct
GESLFKGPRDYNPISSTICHLTNESDGHTTSLYGIGFGPFIITNKHLFVRNNGTLVVQSLHGVFKVKNTTTLQQHPIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 4 - protein, 237 aa, synthetic construct
GESLFKGPRDYNPISSTICHLTNESDGHTTSLYGIGFGPFIITNKHLFFRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 5 - protein, 237 aa, synthetic construct
GESLFKGPRDYNPISSTICHLTNESDGHTTSLYGIGFGPFIITNKHLFVRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 6 - protein, 237 aa, synthetic construct
GESLFKGPRDYNPISSTICHLTNESDGHTTSLYGIGFGPFIITNKHLFRRNNGTLVVQSLHGVFKVKNTTTLQQHPIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 7 - protein, 237 aa, synthetic construct
GESLFKGPRDYNPISSTICHLTNESDGHTTSLYGIGFGPFIITNKHLFRRNNGTLVVQSLHGVFKVKNTTTLQQHLIDGRDMIIIRMPPDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 8 - protein, 237 aa, synthetic construct
GESLFKGPRDYNPISSTICHLTNESDGHTTSLYGIGFGPFIITNKHLFRRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTPVPKNFMELLTNQEAQQWVSGWRLNADSVLWGGHKVFMVKPEEPFQPVKEATQLMNE

SEQ ID NO: 9 - protein, 237 aa, synthetic construct
GESLFKGPRDYNPISSTICHLTNESDGHTTSLYGIGFGPFIITNQHLFRRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 10 - protein, 237 aa, synthetic construct
GESLFKGPRDYNPISSTICHLTNESDGHTTSLYGIGFGPFIITNRHLFRRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 11 - protein, 245 aa, synthetic construct
GHHHHHHHGESLFKGPRDYNPISSTICHLTNESDGHTTSLYGIGFGPFIITNKHLFFRNNGTLVVQSLHGVFKVKNTTTLQQHPIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 12 - protein, 245 aa, synthetic construct
GHHHHHHHGESLFKGPRDYNPISSTICHLTNESDGHTTSLYGIGFGPFIITNKHLFVRNNGTLVVQSLHGVFKVKNTTTLQQHPIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 13 - protein, 245 aa, synthetic construct
GHHHHHHHGESLFKGPRDYNPISSTICHLTNESDGHTTSLYGIGFGPFIITNKHLFFRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 14 - protein, 245 aa, synthetic construct
GHHHHHHHGESLFKGPRDYNPISSTICHLTNESDGHTTSLYGIGFGPFIITNKHLFVRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 15 - protein, 245 aa, synthetic construct
GHHHHHHHGESLFKGPRDYNPISSTICHLTNESDGHTTSLYGIGFGPFIITNKHLFRRNNGTLVVQSLHGVFKVKNTTTLQQHPIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 16 - protein, 245 aa, synthetic construct
GHHHHHHHGESLFKGPRDYNPISSTICHLTNESDGHTTSLYGIGFGPFIITNKHLFRRNNGTLVVQSLHGVFKVKNTTTLQQHLIDGRDMIIIRMPPDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 17 - protein, 245 aa, synthetic construct
GHHHHHHHGESLFKGPRDYNPISSTICHLTNESDGHTTSLYGIGFGPFIITNKHLFRRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTPVPKNFMELLTNQEAQQWVSGWRLNADSVLWGGHKVFMVKPEEPFQPVKEATQLMNE

SEQ ID NO: 18 - protein, 245 aa, synthetic construct
GHHHHHHHGESLFKGPRDYNPISSTICHLTNESDGHTTSLYGIGFGPFIITNQHLFRRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 19 - protein, 245 aa, synthetic construct
GHHHHHHHGESLFKGPRDYNPISSTICHLTNESDGHTTSLYGIGFGPFIITNRHLFRRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 20 - protein, 237 aa, synthetic construct
GESLFKGPRDYNPISSTICHLTNESDGHTTSLYGIGFGPFIITNKHLFFRNNGTLLVQSLHGVFKVKNTTTLQQHPIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNE

SEQ ID NO: 21 - protein, 237 aa, synthetic construct
GESLFKGPRDYNPISSTICHLTNESDGHTTSLYGIGFGPFIITNKHLFVRNNGTLLVQSLHGVFKVKNTTTLQQHPIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNE

SEQ ID NO: 22 - protein, 237 aa, synthetic construct
GESLFKGPRDYNPISSTICHLTNESDGHTTSLYGIGFGPFIITNKHLFFRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNE

SEQ ID NO: 23 - protein, 237 aa, synthetic construct
GESLFKGPRDYNPISSTICHLTNESDGHTTSLYGIGFGPFIITNKHLFVRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNE

SEQ ID NO: 24 - protein, 237 aa, synthetic construct
GESLFKGPRDYNPISSTICHLTNESDGHTTSLYGIGFGPFIITNKHLFRRNNGTLLVQSLHGVFKVKNTTTLQQHPIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNE

SEQ ID NO: 25 - protein, 237 aa, synthetic construct
GESLFKGPRDYNPISSTICHLTNESDGHTTSLYGIGFGPFIITNKHLFRRNNGTLLVQSLHGVFKVKNTTTLQQHLIDGRDMIIIRMPPDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNE

SEQ ID NO: 26 - protein, 237 aa, synthetic construct
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTPVPKNFMELLTNQEAQQWVSGWRLNADSVLWGGHKVFMSKPEEPFQPVKEATQLMNE

SEQ ID NO: 27 - protein, 237 aa, synthetic construct
GESLFKGPRDYNPISSTICHLTNESDGHTTSLYGIGFGPFIITNQHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNE

SEQ ID NO: 28 - protein, 237 aa, synthetic construct
GESLFKGPRDYNPISSTICHLTNESDGHTTSLYGIGFGPFIITNRHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNE

SEQ ID NO: 29 - protein, 237 aa, synthetic construct
GESLFKGPRDYNPISSTICHLTNESDGHTTSLYGIGFGPFIITNKHLFRRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 30 - protein, 245 aa, synthetic construct
GHHHHHHHGESLFKGPRDYNPISSTICHLTNESDGHTTSLYGIGFGPFIITNKHLFRRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNE

SEQ ID NO: 31 - protein, 237 aa, Tobacco etch virus
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNE

SEQ ID NO: 32 - protein, 12 aa, synthetic construct
  VARIANT 1..6: note = TEV protease recognition sequence with cleavage between positions 5 and 6
ENLYQGIVKQXL

SEQ ID NO: 33 - protein, 8 aa, synthetic construct
  VARIANT 1: note = E joined to a chitin binding domain
  VARIANT 7: note = 7 is G joined to 6-phospho-B-glucosidase A
ENLYFQGA

SEQ ID NO: 34 - protein, 6 aa, synthetic construct
  VARIANT 5..6: note = Wildtype TEV protease cleaves between 5 and 6 (X=G); optionally X=G, S, A, M, C, N, Y, K, D, Q, F, T, W, R, L, E, I, or V
ENLYQX

SEQ ID NO: 35 - protein, 628 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPETWDAVRYNGKLIAYPIAVEALSLIYNKDLLPNPPKTWEDGQVKYGSWIPALDKELKAKGKSALMENLQEPYFTWPLIAADGGYAFKYENGKYDIKDVGVDNAGAKAGLTELVDLIKNKHMNADTDYSIAEAAENKGETAMTINGPWASNIDTSKVNYGVIVLPTEKGQPSKPEVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGENLYFQGNSAFGSMSVADLGSLTRLEDKIRLLQEDLESERELRNRIERERADLSVQLIALTDRLEDAEGTTDSQIESNRKREAELQKIRKLLEESQLENEDAMNVLRKKHQDACLDYAEQIEQLQKKNSKIDRERQRLQHEVIELTATIDQLQKDKHLAEKAAERFEAQTIELSNKVEDLNRHVNDLAQQRQRLQAENNDLLKETHDQKVQLDNLQHVKYQLAQQLEEARRRIRIPCR

(19) United States
(12) Patent Application Publication
(10) Pub. No.: US 2026/0234592 A1
(43) Pub. Date: Aug. 13, 2026

(54) COLD-ACTIVE TEV PROTEASES, COMPOSITIONS AND METHODS

(71) Applicant: New England Biolabs, Inc., Ipswich, MA (US)

(72) Inventors: Haruichi Asahara, Ipswich, MA (US); James C. Samuelson, Newburyport, MA (US)

(73) Assignee: New England Biolabs, Inc., Ipswich, MA (US)

(21) Appl. No.: 19/564,439

(22) Filed: Mar. 12, 2026

Related U.S. Application Data
(63) Continuation of application No. PCT/US24/46350, filed on Sep. 12, 2024.
(60) Provisional application No. 63/582,694, filed on Sep. 14, 2023.

(51) Int. Cl. C12N 9/50; C12P 21/06

(52) U.S. Cl. CPC C12N 9/506; C12P 21/06; C12Y 304/22044

(57) ABSTRACT

The present disclosure relates, according to some embodiments, to compositions, methods and kits for cleaving a target protein using a cold-active variant TEV protease.

### CROSS-REFERENCE TO RELATED APPLICATIONS

This application is a continuation of International Application No. PCT/US2024/046350, filed Sep. 12, 2024, which claims the benefit of U.S. Provisional Application Ser. No. 63/582,694, filed Sep. 14, 2023. The contents of all of the above are hereby incorporated in their entirety by reference.

### SEQUENCE LISTING STATEMENT

This disclosure includes a Sequence Listing submitted electronically in .xml format under the file name “NEB-473.xml” created on Mar. 12, 2026, and having a size of 47,1564 bytes. This Sequence Listing is incorporated herein in its entirety by this reference.

### BACKGROUND

Catalytic activity of proteases, like other enzymes, tends to increase with increasing temperature and decrease with decreasing temperatures. For example, many enzymes display 50% to 100% higher activity when the reaction temperature increases by 10° C. Significant changes in activity (e.g., 10%, 20%) may be observed when the reaction temperatures changed by 1° C. or 2° C. This correlation between activity and reaction temperature may be limited to a range of reaction temperatures, for example, from 15° C. to 45° C. Higher reaction temperatures may see catalytic activity peak and then fall, potentially as a result of protein denaturation while lower temperatures may see catalytic activity may slow or even fall to zero, potentially as a result of the enzyme becoming too rigid to interact with a substrate, execute the reaction, and/or release product(s).

Proteases, including peptidases, are found ubiquitously in prokaryotes and eukaryotes. For example, at least 14 superfamilies of cysteine proteases have been described, each containing many families of proteins. Endopeptidases are proteases (proteolytic peptidases) that cleave peptide bonds between internal amino acids in proteins. Tobacco Etch Virus (TEV) protease is a highly specific cysteine protease used for the removal of affinity purification tags such as maltose-binding protein (MBP) or poly-histidine from fusion proteins. For example, TEV protease may be used to cleave an affinity tag from a fusion comprising the affinity tag and a protein of interest (e.g., an enzyme). TEV protease may have peak activity at ~30° C., but proteins of interest may lack stability at such temperatures. At lower temperatures, where proteins of interest are (more) stable, TEV protease may have reduced or no activity.

### SUMMARY

Accordingly, needs have arisen for endopeptidases with improved activity at low temperatures. The present disclosure relates to systems, apparatus, compositions, enzymes, and/or methods for cleaving proteins of interest at low temperatures. Cold-active proteases, according to some embodiments, may be more efficient at cold temperatures than at warmer temperatures and/or more efficient than other proteases at the same cold temperature. A cold-active variant TEV protease may have, for example, an amino acid sequence at least 90% identical to SEQ ID NO:1, wherein X49 is any amino acid other than arginine, X73 is any amino acid other than glutamine, X76 is any amino acid other than leucine, X78 is any amino acid other than aspartate, X82 is any amino acid other than methionine, X83 is any amino acid other than isoleucine, X84 is any amino acid other than isoleucine, X89 is any amino acid other than lysine, X102 is any amino acid other than glutamate, X150 is any amino acid other than glutamine, X155 is any amino acid other than leucine, X181 is any amino acid other than serine, X184 is any amino acid other than lysine, X193 is any amino acid other than glutamine, X206 is any amino acid other than alanine, or X223 is any amino acid other than glutamate. In some embodiments, a cold-active variant TEV protease may have an amino acid sequence at least 90% identical to SEQ ID NO:1, wherein at least one of X49, X73, X76, X78, X82, X83, X84, X89, X102, X150, X155, X181, X184, X193, X206, and X223 constitutes a substitution relative to the corresponding position of SEQ ID NO:31. For example, a cold-active variant TEV protease may have an amino acid sequence at least 98% identical to SEQ ID NO:2 or 3 or at least 97% identical to SEQ ID NO:20 or 21 or at least 95% identical to SEQ ID NO:11 or 12. In some embodiments, (a) X49 may be any amino acid other than arginine or (b) X76 may be any amino acid other than leucine or (c) X49 may be any amino acid other than arginine and X76 may be any amino acid other than leucine. For example, (a) X49 may be phenylalanine or valine or (b) X76 may be proline or (c) X49 may be phenylalanine or valine and X76 may be proline. A cold-active variant TEV protease may have an amino acid sequence comprising one or more substitutions at position 45, a substitution at position 56, and a substitution at position 219 (in each case, relative to the corresponding position of SEQ ID NO:31). A cold-active variant TEV protease, according to some embodiments, may have an amino acid sequence comprising one or more of a K45Q substitution, a K45R substitution, a L56V substitution, a S135G substitution, and a S219V substitution. In some embodiments, a cold-active variant TEV protease may have an amino acid sequence, wherein the amino acid sequence is identical to SEQ ID NO:1 at one or more of positions 2, 3, 12, 17, 23, 28, 30, 45, 50, 56, 58, 67, 68, 74, 77, 79, 80, 87, 90, 93, 106, 107, 110, 119, 120, 122, 127, 130, 132, 135, 138, 141, 146, 147, 148, 151, 153, 158, 162, 168, 170, 171, 173, 176, 177, 180, 203, 205, 209, 211, 215, 216, 217, 218, 219, 220, 224, 225, 226, 227, 228, 229, 235, 236, and 237.

According to some embodiments, a cold-active variant TEV protease may comprise an amino acid sequence having (a) at least 85%, at least 90%, or at least 95% identity to SEQ ID NO: 1, (b) at least one substitution relative to SEQ ID NO:31 at a position corresponding to position 49, 73, 76, 78, 82, 83, 84, 89, 102, 150, 155, 181, 184, 193, 206, 223 of SEQ ID NO:31, (c) optionally a substitution at any of its positions or any combination of its positions corresponding to positions 56, 135, and 219 of SEQ ID NO:31, and (d) optionally identity to wildtype TEV protease at any of its positions or any combination of its positions corresponding to positions 2, 3, 12, 17, 23, 28, 30, 45, 50, 56, 58, 67, 68, 74, 77, 79, 80, 87, 90, 93, 106, 107, 110, 119, 120, 122, 127, 130, 132, 135, 138, 141, 146, 147, 148, 151, 153, 158, 162, 168, 170, 171, 173, 176, 177, 180, 203, 205, 209, 211, 215, 216, 217, 218, 219, 220, 224, 225, 226, 227, 228, 229, 235, 236, 237 of SEQ ID NO:31.

A cold-active variant TEV protease may comprise an amino acid sequence, in some embodiments, (a) having at least 85%, at least 90%, or at least 95% identity to SEQ ID NO:1, (b) having at least one substitution relative to wildtype TEV protease (SEQ ID NO:31) at a position corresponding to position 49, 73, 76, 78, 82, 83, 84, 89, 102, 150, 155, 181, 184, 193, 206, 223 of SEQ ID NO:31, (c) optionally having a substitution at any of its positions or any combination of its positions corresponding to positions 56, 135, and 219 of SEQ ID NO:31, (d) optionally lacking one or more of the following substitutions at its positions corresponding to: E2K, S3P, N12D, T17S, N23Q, H28L, H28Y, T30A, T30I, K45F, K45W, R50G, L56V, Q58F, Q58Y, Q58I, K67E, N68D, Q74L, I77V, G79E, R80S, M87L, M87T, D90G, P93S, E106G, E107D, C110S, K119E, S120R, S122P, D127A, C130S, F132L, F132S, S135G, S135F, I138T, K141R, T146A, T146C, T146S, K147E, D148R, D148P, D148A, C151A, S153C, S153N, T158A, F162S, F162A, S168T, S170A, N171D, N171Q, T173A, T173G, N176I, N176T, N177K, N177R, N177S, N177M, N177Y, T180A, R203Q, N205D, V209M, V209F, W211I, W211V, W211L, W211C, K215E, V216I, F217K, M218I, M218F, M218W, M218L, M218T, S219D, S219E, S219V, S219P, S219N, K220R, P224S, F225L, Q226stop, Q226S, Q226P, P227A, V228S, K229E, K229stop, M235K, N236S, and E237G of SEQ ID NO:31; and (e) optionally lacking one or more of the following substitutions at its positions corresponding to: K67P, M82I, 183V, Q150D, Q150H, S153L, L155A, L155M, and G213P of SEQ ID NO:31.

A cold-active variant TEV protease, according to some embodiments, may have an amino acid sequence comprising a K45Q substitution or a K45R substitution and, optionally, one or more additional substitutions (e.g., a L56V substitution, a S135G substitution, and a S219V substitution), in each case, the substitutions being at a position corresponding to a wild type sequence. A cold-active TEV protease may have, for example, at least 90%, at least 92%, at least 95%, at least 97%, or at least 98% identity to one or more of SEQ ID NOS: 9, 10, 18, 19, 27 and 28. For example, a cold-active TEV protease may have an amino acid sequence at least 98% identical to SEQ ID NO:9 or 10 or at least 97% identical to SEQ ID NO:27 or 28 or at least 95% identical to SEQ ID NO:18 or 19.

Provided variant TEV proteases may be more efficient (e.g., more catalytically efficient) than control TEV proteases. For example, the ratio of the k_{cat}/K_{M} of a variant TEV protease (e.g., SEQ ID NOS: 1-28) to the k_{cat}/K_{M} of a control TEV protease (e.g., SEQ ID NOS: 29-30) may be in a range of 1.3-3.0.

The present disclosure further relates to methods of cleaving one or more TEV protease substrates. A method may include, for example, (a) contacting (i) a TEV protease substrate comprising a TEV protease recognition sequence having the amino acid sequence of SEQ ID NO: 34, a protein of interest attached to the TEV protease recognition sequence, and a removable segment attached to the TEV protease recognition sequence, and (ii) a variant TEV protease (e.g., any of the variant TEV proteases disclosed herein) to form a cleavage product mixture comprising separately the protein of interest and the removable segment; and optionally separating the protein of interest from the removable segment, wherein the contacting may be performed at a temperature below 20° C., below 18° C., below 16° C., below 14° C., below 12° C., below 10° C., below 8° C., below 6° C., below 4° C., below 2° C. or below 0° C.

In some embodiments, a method may comprise contacting (a) composition comprising or potentially comprising a protein comprising a TEV protease recognition sequence having the amino acid sequence of SEQ ID NO:34, and (b) a variant TEV protease (e.g., any of the variant TEV proteases disclosed herein) to produce a cleavage product mixture, wherein the contacting is performed at a temperature below 20° C., below 18° C., below 16° C., below 14° C., below 12° C., below 10° C., below 8° C., below 6° C., below 4° C., below 2° C. or below 0° C., and/or wherein the cleavage product mixture optionally (i) is free of the intact protein comprising a TEV protease recognition sequence (e.g., becomes free of the intact protein in ≤3 hours, ≤5 hours, ≤12 hours, or ≤18 hours of the contacting), and/or (ii) comprises at least two fragments of the protein comprising the TEV protease recognition sequence (e.g., in ≤1 hour, ≤3 hours, ≤5 hours, or ≤12 hours of the contacting).

A method may comprise (a) contacting (i) a TEV protease substrate comprising a TEV protease recognition sequence having the amino acid sequence of SEQ ID NO:34, a support attached to the TEV protease recognition sequence, and a bait molecule attached to the TEV protease recognition sequence, and (ii) a composition comprising a target molecule capable of binding the bait molecule to form one or more TEV protease substrate: target molecule complexes. A method may comprise (e.g., may further comprise) (b) contacting the one or more TEV protease substrate: target molecule complexes with a variant TEV protease (e.g., any of the variant TEV proteases disclosed herein) to cleave the TEV protease substrate at the recognition sequence. The (a) contacting may be performed, the (b) contacting may be performed, or the (a) contacting and the (b) contacting may both be performed a temperature below 20° C., below 18° C., below 16° C., below 14° C., below 12° C., below 10° C., below 8° C., below 6° C., below 4° C., below 2° cor below 0° C.

Provided methods may use any desired variant TEV protease disclosed herein including, for example, variant TEV proteases that are more efficient (e.g., more catalytically efficient) than control TEV proteases. For example, the ratio of the k_{cat}/K_{M} of a variant TEV protease (e.g., any of SEQ ID NOS: 1-28) to the k_{cat}/K_{M} of a control TEV protease (e.g., SEQ ID NO: 29 or 30) may be in a range of 1.3-3.0. Variant TEV proteases to be used in disclosed methods, compositions, or kits may have any desired form including, for example, a soluble form, a lyophilized form, or an immobilized form.

The present disclosure further relates to kits for cleaving a target protein (e.g., any protein of interest having a TEV protease recognition sequence). A kit, according to some embodiments, may comprise a variant TEV protease (e.g., any of the variant TEV proteases disclosed herein) and a buffer (e.g., a reaction buffer or a storage buffer), wherein the variant TEV protease has a form selected from a liquid form, a gel form, an aqueous form, a film form, a crystalline form, a powder form, a cake form, a dried form, freeze dried form, a lyophilized form, and an immobilized form.

### BRIEF DESCRIPTION OF THE FIGURES

FIG. 1 shows an example correlation between concentration of a TEV protease and fluorescence representing cleavage activity of a peptide substrate.

FIG. 2 shows SDS-PAGE fractionation of example products of a cell-free protein synthesis system. The arrow marks the expected location of the example TEV protease variants indicated above each lane.

FIG. 3 shows an example of activity assay results obtained with some TEV protease variants comprising proline substitutions. Arrows mark variants that displayed higher activity than the control (a TEV protease having substitutions L56V, S135G, and S219V). Some variants displayed comparable activity to the control (e.g., variants comprising K184P, Q193P, A206P, or E223P).

FIG. 4 shows an example of activity assay results obtained with some TEV protease variants comprising substitutions near His46. Arrows mark variants that displayed higher activity than the control.

FIG. 5 shows an example of activity assay results obtained with some TEV protease variants comprising single or double substitutions. Arrows mark variants that displayed higher activity than the control.

FIG. 6 shows a time course of CBD-TEVrs-bglA cleavage by some example TEV protease variants at 30° C.

FIG. 7 shows a time course of CBD-TEVrs-bglA cleavage by some example TEV protease variants at 20° C.

FIG. 8 shows example CBD-TEVrs-bglA cleavage results obtained with some example TEV protease variants after 18 hours at 4° C.

FIG. 9 shows an example of activity assay results obtained with some TEV protease variants comprising double substitutions. Arrows mark variants having a hydrophobic amino acid at position 49.

FIG. 10 shows a time course of cleavage results obtained with some example TEV protease variants at 30° C.

FIG. 11 shows example CBD-TEVrs-bglA cleavage results obtained with 4 μM to 0.03125 μM (2-fold serial) of control and R49F_L76P variant TEV protease after 18 hours at 4° C. Under the conditions tested, complete cleavage was observed at 2 μM control TEV protease and at 0.5 μM R49F_L76P variant TEV protease.

FIG. 12 shows example CBD-TEVrs-bglA cleavage results obtained with some example TEV protease variants after 18 hours at 4° C. (lanes 3, 4, and 5) or 0° C. (lanes 6, 7, and 8). Percentage substrate cleavage is shown below each lane.

FIGS. 13A-13D show example correlations between variant TEV proteases at selected concentrations and cleavage ratios after 18 hours at 4° C. with CBD-TEVrs-bglA. FIG. 13A shows results for the R49V variant TEV protease. FIG. 13B shows results for the L76P variant TEV protease. FIG. 13C shows results for the R49V_L76P variant TEV protease. FIG. 13D shows results for the R49F_L76P variant TEV protease.

FIGS. 14A-14D show example kinetic analyses of TEV protease variants relative to control TEV protease using the data of FIGS. 13A-13D, respectively. FIG. 14A shows a kinetic analysis of the R49V variant in which the k_{cat}/K_{M} ratio was 2.0. FIG. 14B shows a kinetic analysis of the L76P variant in which the k_{cat}/K_{M} ratio was 1.7. FIG. 14C shows a kinetic analysis of the R49V_L76P variant in which the k_{cat}/K_{M} ratio was 1.5. FIG. 14D shows a kinetic analysis of the R49F_L76P variant in which the k_{cat}/K_{M} ratio was 2.6.

FIG. 15 shows example MBP5-TEV-paramyosin-ΔSal cleavage results obtained with 0.125 μg of a commercially available TEV protease (“Std”; New England Biolabs, Inc. Catalog #P8112S) and an R49F_L76P variant (“Cold”; SEQ ID NO:11) TEV protease after the indicated times (in hours TO, T1, T3, and T5) at 4° C.

### BRIEF DESCRIPTION OF THE SEQUENCES

Some embodiments of this disclosure relate to the following provided sequences of example polynucleotides and/or example polypeptides.

SEQ ID NO: 1 is an example TEV protease variant having at least one substitution at a position that corresponds to a position of SEQ ID NO:31 selected from positions 45, 49, 73, 76, 78, 82, 83, 84, 89, 102, 150, 155, 181, 184, 193, 206, and 223. Each X is independently any amino acid except that at least one of the following is true: 45 is not K, 49 is not R, 73 is not Q, 76 is not L, 78 is not D, 82 is not M, 83 is not I, 84 is not I, 89 is not K, 102 is not E, 150 is not Q, 155 is not L, 181 is not S, 184 is not K, 193 is not Q, 206 is not A, and/or 223 is not E. Although not marked with “X”, optionally, 56 is not L (e.g., is V instead of L), 135 is not S (e.g., is G instead of S), and/or 219 is not S (e.g., is V instead of S).

SEQ ID NO:2 is an example TEV protease variant having substitutions at positions that correspond to SEQ ID NO:31 positions 49 (R49F), 56 (L56V), 76 (L76P), 135 (S135G), and 219 (S219V). Enzymes comprising this sequence may be labeled “R49F_L76P”.

SEQ ID NO:3 is an example TEV protease variant having substitutions at positions that correspond to SEQ ID NO:31 positions 49 (R49V), 56 (L56V), 76 (L76P), 135 (S135G), and 219 (S219V). Enzymes comprising this sequence may be labeled “R49V_L76P”.

SEQ ID NO:4 is an example TEV protease variant having substitutions at positions that correspond to SEQ ID NO:31 positions 49 (R49F), 56 (L56V), 135 (S135G), and 219 (S219V). Enzymes comprising this sequence may be labeled “R49F”.

SEQ ID NO:5 is an example TEV protease variant having substitutions at positions that correspond to SEQ ID NO:31 positions 49 (R49V), 56 (L56V), 135 (S135G), and 219 (S219V). Enzymes comprising this sequence may be labeled “R49V”.

SEQ ID NO:6 is an example TEV protease variant having substitutions at positions that correspond to SEQ ID NO:31 positions 56 (L56V), 76 (L76P), 135 (S135G), and 219 (S219V). Enzymes comprising this sequence may be labeled “L76P”.

SEQ ID NO:7 is an example TEV protease variant having substitutions (relative to the corresponding position of SEQ ID NO:31) at positions that correspond to SEQ ID NO:31 positions 56 (L56V), 89 (K89P), 135 (S135G), and 219 (S219V). Enzymes comprising this sequence may be labeled “K89P”.

SEQ ID NO:8 is an example TEV protease variant having substitutions (relative to the corresponding position of SEQ ID NO:31) at positions that correspond to SEQ ID NO:31 positions 56 (L56V), 135 (S135G), 181 (S181P), and 219 (S219V). Enzymes comprising this sequence may be labeled “S181P”.

SEQ ID NO:9 is an example TEV protease variant having substitutions (relative to the corresponding position of SEQ ID NO:31) at positions that correspond to SEQ ID NO:31 positions 45 (K45Q), 56 (L56V), 135 (S135G), and 219 (S219V). Enzymes comprising this sequence may be labeled “K45Q”.

SEQ ID NO:10 is an example TEV protease variant having substitutions (relative to the corresponding position of SEQ ID NO:31) at positions that correspond to SEQ ID NO:31 positions 45 (K45R), 56 (L56V), 135 (S135G), and 219 (S219V). Enzymes comprising this sequence may be labeled “K45R”.

SEQ ID NO:11 is an example TEV protease variant having an N-terminal His tag and substitutions at positions that correspond to SEQ ID NO:31 positions 49 (R49F), 56 (L56V), 76 (L76P), 135 (S135G), and 219 (S219V). Enzymes comprising this sequence may be labeled “R49F_L76P”.

SEQ ID NO:12 is an example TEV protease variant having an N-terminal His tag and substitutions at positions that correspond to SEQ ID NO:31 positions 49 (R49V), 56 (L56V), 76 (L76P), 135 (S135G), and 219 (S219V). Enzymes comprising this sequence may be labeled “R49V_L76P”.

SEQ ID NO:13 is an example TEV protease variant having an N-terminal His tag and substitutions at positions that correspond to SEQ ID NO:31 positions 49 (R49F), 56 (L56V), 135 (S135G), and 219 (S219V). Enzymes comprising this sequence may be labeled “R49F”.

SEQ ID NO:14 is an example TEV protease variant having an N-terminal His tag and substitutions at positions that correspond to SEQ ID NO:31 positions 49 (R49V), 56 (L56V), 135 (S135G), and 219 (S219V). Enzymes comprising this sequence may be labeled “R49V”.

SEQ ID NO:15 is an example TEV protease variant having an N-terminal His tag and substitutions at positions that correspond to SEQ ID NO:31 positions 56 (L56V), 76 (L76P), 135 (S135G), and 219 (S219V). Enzymes comprising this sequence may be labeled “L76P”.

SEQ ID NO:16 is an example TEV protease variant having an N-terminal His tag and substitutions at positions that correspond to SEQ ID NO:31 positions 56 (L56V), 89 (K89P), 135 (S135G), and 219 (S219V). Enzymes comprising this sequence may be labeled “K89P”.

SEQ ID NO:17 is an example TEV protease variant having an N-terminal His tag and substitutions at positions that correspond to SEQ ID NO:31 positions 56 (L56V), 135 (S135G), 181 (S181P), and 219 (S219V). Enzymes comprising this sequence may be labeled “S181P”.

SEQ ID NO:18 is an example TEV protease variant having an N-terminal His tag and substitutions at positions that correspond to SEQ ID NO:31 positions 45 (K45Q), 56 (L56V), 135 (S135G), and 219 (S219V). Enzymes comprising this sequence may be labeled “K45Q”.

SEQ ID NO:19 is an example TEV protease variant having an N-terminal His tag and substitutions at positions that correspond to SEQ ID NO:31 positions 45 (K45R), 56 (L56V), 135 (S135G), and 219 (S219V). Enzymes comprising this sequence may be labeled “K45R”.

SEQ ID NO:20 is an example TEV protease variant having substitutions at positions that correspond to SEQ ID NO:31 positions 49 (R49F) and 76 (L76P). Enzymes comprising this sequence may be labeled “R49F_L76P”. [WT@ 56 (L56), 135 (S135), and 219 (S219).]

SEQ ID NO:21 is an example TEV protease variant having substitutions at positions that correspond to SEQ ID NO:31 positions 49 (R49V) and 76 (L76P). Enzymes comprising this sequence may be labeled “R49V_L76P”. [WT@ 56 (L56), 135 (S135), and 219 (S219).]

SEQ ID NO:22 is an example TEV protease variant having a substitution at the position corresponding to position 49 (R49F) of SEQ ID NO:31. Enzymes comprising this sequence may be labeled “R49F”. [WT@ 56 (L56), 135 (S135), and 219 (S219).]

SEQ ID NO:23 is an example TEV protease variant having a substitution at the position corresponding to position 49 (R49V) of SEQ ID NO:31. Enzymes comprising this sequence may be labeled “R49V”. [WT@ 56 (L56), 135 (S135), and 219 (S219).]

SEQ ID NO:24 is an example TEV protease variant having a substitution at the position corresponding to position 76 (L76P) of SEQ ID NO:31. Enzymes comprising this sequence may be labeled “L76P”. [WT@ 56 (L56), 135 (S135), and 219 (S219).]

SEQ ID NO:25 is an example TEV protease variant having a substitution at the position corresponding to position 89 (K89P) of SEQ ID NO:31. Enzymes comprising this sequence may be labeled “K89P”. [WT@ 56 (L56), 135 (S135), and 219 (S219).]

SEQ ID NO:26 is an example TEV protease variant having a substitution at the position corresponding to position 181 (S181P) of SEQ ID NO:31. Enzymes comprising this sequence may be labeled “S181P”. [WT@ 56 (L56), 135 (S135), and 219 (S219).]

SEQ ID NO:27 is an example TEV protease variant having a substitution at the position corresponding to position 45 (K45Q) of SEQ ID NO:31. Enzymes comprising this sequence may be labeled “K45Q”. [WT@ 56 (L56), 135 (S135), and 219 (S219).]

SEQ ID NO:28 is an example TEV protease variant having a substitution at the position corresponding to position 45 (K45R) of SEQ ID NO:31. Enzymes comprising this sequence may be labeled “K45R”. [WT@ 56 (L56), 135 (S135), and 219 (S219).]

SEQ ID NO:29 is an example TEV protease variant having substitutions at positions that correspond to SEQ ID NO:31 positions 56 (L56V), 135 (S135G), and 219 (S219V). In some embodiments, enzymes comprising this sequence are labeled “control”.

SEQ ID NO:30 is an example TEV protease variant having an N-terminal His tag and substitutions at positions that correspond to SEQ ID NO:31 positions 56 (L56V), 135 (S135G), and 219 (S219V). Enzymes comprising this sequence may be labeled “control”.

SEQ ID NO:31 is an example wildtype TEV protease. One or more of positions 45, 49, 73, 76, 78, 82, 83, 84, 89, 102, 150, 155, 181, 184, 193, 206, 223 may be substituted as disclosed herein.

SEQ ID NO:32 is an example fluorogenic peptide (TEV protease substrate) having an example recognition sequence at positions 1-6, wherein cleavage occurs between positions 5 and 6.

SEQ ID NO:33 is an example TEV protease substrate, wherein position 1 (E1) further comprises a chitin binding domain and position 7 (G7) is joined to a 6-phospho-β-glucosidase A. In some embodiments, this substrate is labeled “CBD-TEVrs-bglA”.

SEQ ID NO:34 is an example TEV protease recognition sequence, wherein cleavage occurs between positions Q5 and X6. X6 is glycine or optionally X6 may be any of glycine, serine, alanine, methionine, cysteine, asparagine, tyrosine, lysine, aspartate, glutamine, phenylalanine, threonine, tryptophan, arginine, leucine, glutamate, isoleucine, or valine.

SEQ ID NO:35 is an example TEV protease substrate (“MBP5-TEV-paramyosin ΔSal”) comprising a maltose binding protein domain and a paramyosin fragment linked by a TEV protease recognition sequence.

### DETAILED DESCRIPTION

Modern biotechnology affords practitioners choices in the manner of protein production as well as the proteins which may be made. Proteins of interest may be manufactured in cell-based workflows (e.g., bacteria, yeast, mammalian, baculovirus/insect cell systems) or cell-free workflows (e.g., PURExpress® and NEBExpress® protein synthesis systems, both from NEB, Inc., Ipswich MA). In both cases, it may be desired to produce the protein in a form that permits separation or purification from other molecules and materials. For example, proteins may be produced as fusions comprising a purification tag (e.g., an affinity tag) and the protein of interest. Following purification and/or any other desired manipulation, the protein of interest may be cleaved away from the tag (or other fusion partner) with an endopeptidase. The present disclosure relates, in some embodiments, to variant endopeptidases. Variant proteases may cleave polypeptides (e.g., fusion proteins) with higher efficiency and/or at lower temperatures than one or more reference proteases.

### General Considerations

Aspects of the present disclosure can be understood in light of the provided descriptions, figures, sequences, embodiments, section headings, and examples, none of which should be construed as limiting the entire scope of the present disclosure in any way. Accordingly, the innovations set forth herein should be construed in view of the full breadth and spirit of the disclosure.

Each of the individual embodiments described and illustrated herein has discrete components and features which can be readily separated from or combined with the components and/or features of any of the other several embodiments without departing from the scope or spirit of the present teachings. Lists of example species within a particular genus may vary in length at different places throughout the disclosure. Species lists shortened for convenience shall not be construed to exclude example species listed elsewhere in the specification. Any recited method can be carried out in the order of events recited or in any other order which is logically possible. Unless otherwise expressly stated to be required herein, each component, feature, and method step disclosed herein is optional and the disclosure contemplates embodiments in which each optional element may be expressly excluded. As such, this statement is intended to serve as antecedent basis for use of such exclusive terminology as “solely,” “only” and the like in connection with the recitation of claim elements or use of a “negative” limitation. It is further intended to serve as antecedent basis for use of such elective terminology as “optionally” and the like in connection with the recitation of one or more claim elements.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents unless the context clearly dictates otherwise. For example, the term “a protein” refers to one or more proteins, i.e., a single protein and multiple proteins.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified.

In the context of the present disclosure, “buffer” and “buffering agent” refer to a chemical entity or composition that itself resists and, when present in a solution, allows such solution to resist changes in pH when such solution is contacted with a chemical entity or composition having a higher or lower pH (e.g., an acid or alkali). Examples of suitable non-naturally occurring buffering agents that may be used in disclosed compositions, kits, and methods include HEPES, MES, MOPS, TAPS, tricine, and Tris. Additional examples of suitable buffering agents that may be used in disclosed compositions, kits, and methods include ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine.

In the context of the present disclosure, “catalytically active” refers to the property of a molecule (e.g., a proteinaceous molecule or macromolecule) to function as a catalyst of one or more chemical reactions relative to one or more substrates and products. A catalytically active TEV protease, for example, hydrolyzes a substrate polypeptide to yield (even if only briefly, for example, in the context of coupled reactions) products comprising at least two fragments of the substrate polypeptide. Catalytic activity of TEV proteases may be assessed using existing techniques applied to one or more model substrates (e.g., SEQ ID NO:32, SEQ ID NO: 33 and SEQ ID NO:35) and/or one or more substrates of interest. For example, effective assays for catalytic activity of TEV proteases may include (1) cleavage of an indicator peptide comprising a fluorophore, a quencher operably linked to the fluorophore to quench fluorescence of the fluorophore, and a TEV protease recognition site disposed between the fluorophore and the quencher, wherein cleavage is assessed by the appearance and/or intensity of fluorescence discharged by the cleaved peptide and (2) cleavage of a fusion protein comprising a first protein, a second protein, and a TEV protease recognition site disposed between the first and second proteins, wherein cleavage is assessed the appearance and/or intensity of bands corresponding to the first and/or second proteins following size fractionation (e.g., SDS-PAGE) of reaction products. Catalytic activity may be assessed with respect to loss of original substrate polypeptide (e.g., percent of original polypeptide remaining), appearance of one or more cleavage products (e.g., fragments of the substrate polypeptide), and/or metrics that serve as a proxy for any of the foregoing.

In the context of the present disclosure, “catalytic efficiency” refers to the rate at which a catalyst (e.g., a variant TEV protease) catalyzes a reaction, expressed as k_{cat} or k_{cat}/K_{M}.

In the context of the present disclosure, “container” refers to a human-made container. A container may comprise one or more walls (e.g., defining an interior volume) and optionally one or more openings. Containers comprising one or more openings may further comprise one or more closures (e.g., a removable closures) for some or all such openings. A closure optionally may comprise an aperture or a septum, for example, to provide fluid communication with a volume of the container and an inserted tube or syringe. Examples of containers include boxes, cartons, bottles, tubes (e.g., test tubes, microcentrifuge tubes), plates (e.g., 96-well, 384-well plates), vials, pipette tips, and ampules. Containers and/or closures may comprise any desired material including paper, plastics, glass, silicone, composites, metals, alloys, or combinations thereof. Containers and/or closures may comprise materials that are compostable, recyclable, and/or sustainable.

In the context of the present disclosure, “cold-active” enzyme refers to an enzyme that is catalytically active at cold temperatures (e.g., temperatures in ranges X to Y, where X is any of −4° C., −2° C., −2° C., 0° C., 2° C., 4° C., and 8° C. and Y is any of 0° C., 2° C., 4° C., 8° C., 10° C., 15° C., 18° C., and 20° C. and X<Y). A cold-active enzyme may or may not have catalytic activity at warmer temperatures. For example, a cold-active enzyme may have catalytic activity (e.g., >0%, ≤25%, ≤50%, ≤75%, ≤100%, ≥110%, ≥125%, ≥150%, ≥175%, or ≥200% of its cold activity) at warmer temperatures (e.g., temperatures in ranges X′ to Y′, where X′ is any of 10° C., 15° C., 18° C., and 20° C. and Y′ is any of 20° C., 25° C., 30° C., 35° C., 40° C., and 45° C. and X′<Y′) under reaction conditions that are otherwise the same as the cold conditions. A cold-active enzyme may perform a reaction at cooler and warmer temperatures with the same, similar, or different kinetics. A cold-active enzyme may perform a reaction by a reaction mechanism that is the same, similar or different at cooler temperatures than warmer temperatures. The mere possibility that an enzyme may display trace activity at lower temperatures (e.g., 0° C.-4° C.) is not enough to be considered a cold-active enzyme if that trace activity depends on overdosing the reaction with the enzyme (e.g., ≥10×, ≥20×, ≥30×, ≥40×, ≥50× what is needed at higher temperatures) and/or depends on using excessively long incubation times (e.g., ≥5×, ≥10× what is needed at higher temperatures).

In the context of the present disclosure and with respect to an amino acid residue or a nucleotide base position, “corresponding to” refers to positions that lie across from one another when sequences are aligned, e.g., by the BLAST algorithm. An amino acid position in a functional or structural motif in one polymerase may correspond to a position within a functionally equivalent functional or structural motif in another polymerase.

In the context of the present disclosure, “fusion” refers to two or more polypeptides, subunits, or proteins covalently joined to one another (e.g., by a peptide bond). For example, a protein fusion may refer to a non-naturally occurring polypeptide comprising a protein of interest covalently joined to a second polypeptide. Examples of a second polypeptide include a reporter protein, a purification tag, and expression tag, a polynucleotide binding protein, an enzyme, a conjugation tag (e.g., a SNAP® tag), and a peptide linker. Unless otherwise disclosed, the protein of interest may be nearer to the N-terminal end or nearer to the C-terminal end than the second polypeptide to which it is joined. A fusion may comprise a non-naturally occurring single polypeptide chain comprising two proteins or two protein domains joined directly to each other by a peptide bond or joined through a peptide linker (e.g., a cleavable or non-cleavable linker). A variant TEV protease may be included in a fusion.

In the context of the present disclosure, “immobilized” refers to covalent attachment of an enzyme to a solid support with or without a linker. Examples of solid supports include beads (e.g., magnetic, agarose, polystyrene, polyacrylamide, chitin). Beads may include one or more surface modifications (e.g., O⁶-benzylguanine, polyethylene glycol) that facilitate covalent attachment and/or activity of an enzyme of interest. For example, a support may comprise a ligand and an enzyme may have a receptor for such ligand or an enzyme may comprise a ligand and a support may comprise a receptor for such ligand. Receptor-ligand binding may be covalent or non-covalent. Non-covalent attachment (e.g., avidin: biotin, chitin: CBP) may be useful in some embodiments, for example, where the level of dissociation of the binding partner is deemed tolerable. A linker may be disposed between a support and an enzyme. For example, linker disposed between a support and an enzyme may have a first covalent bond to the support and a second covalent bond to the enzyme. An immobilized enzyme comprising a ligand-receptor attachment may have a linker disposed between the support and the ligand-receptor attachment, a linker disposed between the enzyme and the ligand-receptor attachment, or both. An immobilized enzyme comprising a linker may also comprise an optional covalent bond directly between the enzyme and the support. A linker may be of any desired length and have any desired range of motion. A peptide linker may comprise one or more repeats (e.g., 1-10 repeats) of glycine-serine.

In the context of the present disclosure, “non-naturally occurring” refers to a molecule (e.g., a polynucleotide, polypeptide, carbohydrate, or lipid) or composition that does not exist in nature. Such a molecule or composition may differ from naturally occurring molecules or compositions in one or more respects. For example, a polymer (e.g., a polynucleotide, polypeptide, or carbohydrate) may differ in the kind and arrangement of the component parts (e.g., nucleotide sequence, amino acid sequence, or sugar molecules). A polymer may differ from a naturally occurring polymer with respect to the molecule(s) to which it is linked. For example, a “non-naturally occurring” polypeptide (e.g., protein) may differ from naturally occurring polypeptides in its secondary, tertiary, or quaternary structure, by having (or lacking) a chemical bond (e.g., a covalent bond including a peptide bond, a phosphate bond, a disulfide bond, an ester bond, and ether bond, and others) to a lipid, a carbohydrate, a second polypeptide (e.g., a fusion protein), or any other molecule. Similarly, a “non-naturally occurring” polynucleotide or nucleic acid may comprise (or lack) one or more other modifications (e.g., an added label or other moiety) to the 5′-end, the 3′ end, and/or between the 5′- and 3′-ends (e.g., methylation) of the nucleic acid. A “non-naturally occurring” molecule or composition may differ from naturally occurring compositions in one or more of the following respects: (a) having components that are not combined in nature, (b) having components in ratios and/or concentrations not found in nature, (c) lacking one or more components otherwise found in naturally occurring molecules or compositions (e.g., a cell-free composition, a chromosome-free composition, a histone-free composition, a polymerase-free composition, a cell membrane-free composition), (d) having a form not found in nature (e.g., dried, freeze dried, lyophilized, crystalline, aqueous, immobilized), and (e) having one or more additional components beyond those found in nature (e.g., a buffering agent, a detergent, a dye, a solvent or a preservative).

In the context of the present disclosure, an amino acid sequence having a percent identity to a reference sequence may be disclosed with or without specifying one or more of the variant positions. For example, if a 100-amino acid polypeptide is disclosed as having an amino acid sequence having at least 90% identity to a 100-amino acid reference sequence, the sequence may differ from the reference sequence at any positions up to 10. If a 100-amino acid polypeptide is disclosed as having an amino acid sequence having at least 90% identity to a 100-amino acid reference sequence and having a K49R substitution, the sequence may differ from the reference sequence as noted at position 49 plus at any other positions up to 9.

In the context of the present disclosure, with reference to an amino acid, “position” refers to the place such amino acid occupies in the primary sequence of a peptide or polypeptide numbered from its amino terminus to its carboxy terminus.

In the context of the present disclosure, “substitution” refers to an amino acid residue at a position in a comparator amino acid sequence that differs with respect to a corresponding position of a reference amino acid sequence, where the comparator and reference sequences are at least 60% identical to each other or at least 70% identical to each other or at least 80% identical to each other. A reference sequence and comparator sequence may have the same length or similar lengths (e.g., differing by ≤12%, ≤5%, ≤1%). A substitute amino acid residue at a position, in addition to differing from the corresponding position of a reference amino acid sequence, may differ from the amino acid at the corresponding position of all naturally-occurring sequences that are at least 60% identical to each other or at least 70% identical to each other or at least 80% identical to the reference sequence. Optionally, a substitute amino acid may have different properties than the amino acid in the corresponding position of the reference sequence. Optionally, a substitute amino acid may have similar properties to the amino acid in the corresponding position of the reference sequence (a “conservative” substitution). For example, a non-polar amino acid (e.g., A, V, L, I, M, W, and F (and optionally C, G, and P) may substitute for another non-polar amino acid, a polar amino acid (e.g., N, Q, S, T, and Y) may substitute for another polar amino acid (e.g., C, D, E, H, K, N, P. Q, R, S, and T), a positively charged amino acid (H, K, and R) may substitute for another positively charged amino acid, and a negatively charged amino acid (e.g., D and E) may substitute for another negatively charged amino acid. A substitute amino acid may be a natural amino acid (e.g., replacing another natural amino acid or a non-natural amino acid). A substitute amino acid may be a non-natural amino acid (e.g., replacing a natural amino acid or another non-natural amino acid).

In the context of the present disclosure, “variant TEV protease” refers to a non-naturally occurring enzyme that hydrolyzes a peptide bond of one or more polypeptide substrates. A variant TEV protease may recognize, bind to, and/or cleave a polypeptide substrate comprising an amino acid sequence, ENLYQX (SEQ ID NO:34), wherein X may be glycine or, optionally, any of serine, alanine, methionine, cysteine, asparagine, histidine, tyrosine, lysine, aspartate, glutamine, phenylalanine, threonine, tryptophan, arginine, leucine, glutamate, isoleucine, or valine and wherein the cleaved peptide bond is between amino acids Q5 and X6. A variant TEV protease may have catalytic activity at and/or following exposure to temperatures in ranges X to Y, where X is any of −4° C., −2° C., −2° C., 0° C., 2° C., 4° C., 8° C. and Y is any of 0° C., 2° C., 4° C., 8° C., 10° C., 15° C., 18° C., 20° C., 25° C., 30° C., 35° C., 40° C., 45° C., 50° C. and X<Y. A variant TEV protease may cleave a polypeptide substrate more efficiently at any given temperature than a reference protease, for example, wildtype TEV protease (SEQ ID NO:31). For example, the increased cleavage efficiency of a variant TEV protease over a wildtype TEV protease at 4° C. may be in a range X″ to Y″, wherein X″ is any of 5%, 10%, 25%, 50%, 75%, or 100% and Y″ is any of 10%, 25%, 50%, 75%, 100%, 250%, 500%, or 1000% (e.g., 5%-25%, 5%-100%, 10%-250%, 25%-1000% and X′″<Y″).

Catalytic activity of a variant TEV protease may persist across a range of salt concentrations, temperatures and/or pH. For example, a variant TEV protease may display catalytic activity under a range of conditions and/or following removal from exposure to such conditions. A variant TEV protease may have catalytic activity at and/or following exposure to a pH from X^{†} to Y^{†}, where X^{†} is any of pH 4, 4.5, 5, 5.5, 6, 6.5, 7, and Y^{†} is any of pH 6.5, 7, 7.5, 8, 8.5, 9, 9.5, 10, 10.5, 11 and X^{†}<Y^{†}.

A variant TEV protease may have an amino acid sequence sharing any desired degree of sequence identity with wildtype TEV protease (SEQ ID NO:31) up to (but excluding) 100% identity (237/237). A variant TEV protease may comprise an amino acid sequence having at least one substitution relative to wildtype TEV protease (e.g., at least two substitutions, at least three substitutions or at least 4 substitutions) at a position corresponding to position 45, 49, 73, 76, 78, 82, 83, 84, 89, 102, 150, 155, 181, 184, 193, 206, 223 of SEQ ID NO:31. Except for such substitution(s), a variant TEV protease may be identical to a wildtype TEV protease (e.g., SEQ ID NO:31). For example, a variant TEV protease may comprise an amino acid sequence having
(a) at least 85%, at least 90%, at least 95%, at least 97% or at least 98% identity to SEQ ID NO:1,
(b) at least one substitution relative to wildtype TEV protease (SEQ ID NO:31) at a position corresponding to position 49, 73, 76, 78, 82, 83, 84, 89, 102, 150, 155, 181, 184, 193, 206, 223 of SEQ ID NO:31,
(c) optionally a substitution at any of its positions or any combination of its positions corresponding to positions 56, 135, and 219 of SEQ ID NO:31, and
(d) optionally identity to wildtype TEV protease at any of its positions or any combination of its positions corresponding to positions 2, 3, 12, 17, 23, 28, 30, 45, 50, 56, 58, 67, 68, 74, 77, 79, 80, 87, 90, 93, 106, 107, 110, 119, 120, 122, 127, 130, 132, 135, 138, 141, 146, 147, 148, 151, 153, 158, 162, 168, 170, 171, 173, 176, 177, 180, 203, 205, 209, 211, 215, 216, 217, 218, 219, 220, 224, 225, 226, 227, 228, 229, 235, 236, 237 of SEQ ID NO:31.
   In some embodiments, it may be desirable to avoid certain substitutions at some positions. For example, a variant TEV protease may comprise an amino acid sequence:
(a) having at least 85%, at least 90%, at least 95%, at least 97% or at least 98% identity to SEQ ID NO:1,
(b) having at least one substitution relative to wildtype TEV protease (SEQ ID NO: 31) at a position corresponding to position 45, 49, 73, 76, 78, 82, 83, 84, 89, 102, 150, 155, 181, 184, 193, 206, 223 of SEQ ID NO:31, wherein, if the at least one substitution includes a substitution at position 45, the substitution at position 45 is selected from K45Q and K45R;
(c) optionally having a substitution at any of its positions or any combination of its positions corresponding to positions 56, 135, and 219 of SEQ ID NO: 31,
(d) optionally lacking one or more of (e.g., two or more of, three or more of, all of) the following substitutions at its positions corresponding to: E2K, S3P, N12D, T17S, N23Q, H28L, H28Y, T30A, T30I, K45F, K45W, R50G, L56V, Q58F, Q58Y, Q58I, K67E, N68D, Q74L, 177V, G79E, R80S, M87L, M87T, D90G, P93S, E106G, E107D, C110S, K119E, S120R, S122P, D127A, C130S, F132L, F132S, S135G, S135F, I138T, K141R, T146A, T146C, T146S, K147E, D148R, D148P, D148A, C151A, S153C, S153N, T158A, F162S, F162A, S168T, S170A, N171D, N171Q, T173A, T173G, N176I, N176T, N177K, N177R, N177S, N177M, N177Y, T180A, R203Q, N205D, V209M, V209F, W211I, W211V, W211L, W211C, K215E, V216I, F217K, M218I, M218F, M218W, M218L, M218T, S219D, S219E, S219V, S219P, S219N, K220R, P224S, F225L, Q226stop, Q226S, Q226P, P227A, V228S, K229E, K229stop, M235K, N236S, and E237G of SEQ ID NO:31; and
(e) optionally lacking one or more of (e.g., two or more of, three or more of, all of) the following substitutions at its positions corresponding to: K67P, M82I, 183V, Q150D, Q150H, S153L, L155A, L155M, and G213P of SEQ ID NO:31.

In some embodiments, a variant TEV protease may have an amino acid sequence having at least 75%, at least 80%, at least 85%, at least 90%, at least 92%, at least 94%, at least 95%, at least 98%, or at least 99% identity to SEQ ID NO: 1, wherein at least one of X45, X49, X73, X76, X78, X82, X83, X84, X89, X102, X150, X155, X181, X184, X193, X206, and X223 constitutes a substitution relative to the corresponding position of SEQ ID NO:31, for example, wherein X45 is any amino acid other than lysine (e.g., K45Q, K45R), wherein X49 is any amino acid other than arginine, X73 is any amino acid other than glutamine, X76 is any amino acid other than leucine, X78 is any amino acid other than aspartate, X82 is any amino acid other than methionine, X83 is any amino acid other than isoleucine, X84 is any amino acid other than isoleucine, X89 is any amino acid other than lysine, X102 is any amino acid other than glutamate, X150 is any amino acid other than glutamine, X155 is any amino acid other than leucine, X181 is any amino acid other than serine, X184 is any amino acid other than lysine, X193 is any amino acid other than glutamine, X206 is any amino acid other than alanine, and/or X223 is any amino acid other than glutamate. A variant TEV protease may optionally include at least one additional substitution (relative to the corresponding position of SEQ ID NO: 31). For example, a variant TEV protease may have an amino acid sequence having one or more substitutions relative to and at positions corresponding to positions 56, 135, 219 of SEQ ID NO:31. A variant TEV protease optionally may further comprise, for example, one or more of the following substitutions relative to SEQ ID NO:31: L56V, S135G, and S219V. A variant TEV protease optionally may further comprise identity to SEQ ID NO:31 at one or more positions corresponding to positions 2, 3, 12, 17, 23, 28, 30, 45, 50, 56, 58, 67, 68, 74, 77, 79, 80, 87, 90, 93, 106, 107, 110, 119, 120, 122, 127, 130, 132, 135, 138, 141, 146, 147, 148, 151, 153, 158, 162, 168, 170, 171, 173, 176, 177, 180, 203, 205, 209, 211, 215, 216, 217, 218, 219, 220, 224, 225, 226, 227, 228, 229, 235, 236, and 237 of SEQ ID NO:31.

According to some embodiments, a variant TEV protease may have at least one substitution relative to wildtype TEV protease (SEQ ID NO:31) and/or may have identity with one or more amino acids of wildtype TEV protease (SEQ ID NO:31). Examples of positions corresponding to wildtype TEV protease (SEQ ID NO:31) that may be identical or have a substitute amino acid (in each case relative to wildtype) are shown in TABLE 1.

TABLE 1

| AA Sequence of Variant | | |
|---|---|---|
| TEV protease may have: | Positions corresponding to listed positions of SEQ ID NO: 31 | |
| Identity at any of (zero | 2, 3, 12, 17, 23, 28, 30, 45, 50, 56, 58, 67, 68, 74, 77, 79, 80, | |
| up to all of) | 87, 90, 93, 106, 107, 110, 119, 120, 122, 127, 130, 132, 135, | |
| | 138, 141, 146, 147, 148, 151, 153, 158, 162, 168, 170, 171, | |
| | 173, 176, 177, 180, 203, 205, 209, 211, 215, 216, 217, 218, | |
| | 219, 220, 224, 225, 226, 227, 228, 229, 235, 236, and/or 237 | |
| Examples include | □ | 17, 28, 30, 68, 107, 127, 132, 135, 146, 148, 153, 162, 170, |
| identity at all | | 171, 176, 177, 209, 211, 218, and 229; |
| positions of each | □ | 12, 68, 74, and 235; |
| bullet | □ | 17, 56, 68, 77, 135, and 219; |
| | □ | 17, 68, and 77; |
| | □ | 17, 68, 77, and 219; |
| | □ | 17 and 80; |
| | □ | 17, 146, 148, 153, 168, 170, 173, and 219; |
| | □ | 23 and 130; |
| | □ | 23, 110, 130, and 173; |
| | □ | 23, 130, and 173; |
| | □ | 23, 130, and 171; |
| | □ | 30, 90, 158, and 237; |
| | □ | 46, 81, and 151; |
| | □ | 56 and 135; |
| | □ | 68, 93, 122, and 135; |
| | □ | 79, 173, and 219; |
| | □ | 120, 148, 173, 177, 218, and 219; |
| | □ | 127, 135, and 176; |
| | □ | 127, 135, 176, 209, 211, and 218; |
| | □ | 127, 135, 146, 148, 176, 177, 209, 211, and 218 |
| | □ | 45, 67, 83, 150, 155, and 213; or |
| | □ | 130 and 173 |
| Substitution(s) at any of | 1*, 45, 49, 56, 73, 76, 77, 78, 82, 83, 84, 87, 89, 102, 135, 150, | |
| (up to all of) its | 153, 155, 181, 184, 193, 206, 219, 223, and/or 237† | |
| Examples include | □ | 45 and 49 (e.g., K45R and R49V); |
| substitutions at all | □ | 45 and 76 (e.g., K45R and L76P); |
| positions of each | □ | 49 and 76 (e.g., R49V and L76P, R49F and L76P); |
| bullet | □ | 49, 56, 76, 135, and 219 (e.g., R49V, L56V, L76P, S135G, |
| | | and S219V; R49F, L56V, L76P, S135G, and S219V) |
| | □ | 45, 56, 135, and 219 (e.g., K45A, L56V, S135G, and |
| | | S219V; K45G, L56V, S135G, and S219V; K45H, L56V, |
| | | S135G, and S219V; K45Q, L56V, S135G, and S219V; |
| | | K45R, L56V, S135G, and S219V; K45S, L56V, S135G, |
| | | and S219V); |
| | □ | 45, 49, 56, 135, and 219 (e.g., K45R, R49V, L56V, S135G, |
| | | and S219V); |
| | □ | 45, 56, 76, 135, and 219 (e.g., K45R, L56V, L76P, S135G, |
| | | and S219V); |
| | □ | 49, 56, 135, and 219 (e.g., R49E, L56V, S135G, and |
| | | S219V; R49K, L56V, S135G, and S219V; R49V, L56V, |
| | | S135G, and S219V); |
| | □ | 49, 56, 76, 135, and 219 (e.g., R49V, L56V, L76P, S135G, |
| | | and S219V); |
| | □ | 49, 73, 76, 78, 82, 83, 84, 89, 102, 150, 155, 181, 184, 193, |
| | | 206, 213, and 223 |
| | □ | 56, 67, 135, and 219 (e.g., L56V, K67P, S135G, and |
| | | S219V); |
| | □ | 56, 73, 135, and 219 (e.g., L56V, Q73P, S135G, and |
| | | S219V); |
| | □ | 56, 76, 135, and 219 (e.g., L56V, L76P, S135G, and |
| | | S219V); |
| | □ | 56, 78, 135, and 219 (e.g., L56V, D78P, S135G, and |
| | | S219V); |
| | □ | 56, 89, 135, and 219 (e.g., L56V, K89P, S135G, and |
| | | S219V); |
| | □ | 56, 102, 135, and 219 (e.g., L56V, E102P, S135G, and |
| | | S219V); |
| | □ | 56, 135, 181, and 219 (e.g., L56V, S135G, S181P, and |
| | | S219V); |
| | □ | 56, 135, 184, and 219 (e.g., L56V, S135G, K184P, and |
| | | S219V); |
| | □ | 56, 135, 193, and 219 (e.g., L56V, S135G, Q193P, and |
| | | S219V); |
| | □ | 56, 135, 206, and 219 (e.g., L56V, S135G, A206P, and |
| | | S219V); |
| | □ | 56, 135, 213, and 219 (e.g., L56V, S135G, G213P, and |
| | | S219V); |
| | □ | 56, 135, and 219 (e.g., L56V, S135G, and S219V); or |
| | □ | 56, 135, 219, and 223 (e.g., L56V, S135G, S219V, and |
| | | E223P) |

*The indicated position may be a single amino acid substitution, an identical amino acid having an N-terminal extension (e.g., a label, a signal peptide, purification tag) or a single amino acid substitution, wherein the single amino acid substitution further comprises an N-terminal extension (e.g., a label, a signal peptide, purification tag).
†The indicated position may be a single amino acid substitution, an identical amino acid having a C-terminal extension (e.g., a label, a peptide, purification tag) or a single amino acid substitution, wherein the single amino acid substitution further comprises a C-terminal extension (e.g., a label, a peptide, purification tag).

In some embodiments, a variant TEV protease may have an amino acid sequence according to any of SEQ ID NOS: 1-30. In some embodiments, a variant TEV protease may have an amino acid sequence according to any of SEQ ID NOS: 1-28. A variant TEV protease fusion may have an amino acid sequence comprising a first portion and a second portion, the first or second portion comprising the variant TEV protease (e.g., the variant TEV protease having any of SEQ ID NOS: 1-28).

A variant TEV protease may have an improved catalytic efficiency over a control TEV protease according to some embodiments. For a given substrate (e.g., CBD-TEVrs-bglA or any other protein comprising a TEVpRS) and at a given temperature (e.g., 4° C.), the ratio of the k_{cat}/K_{M} of a variant TEV protease to the k_{cat}/K_{M} of a control TEV protease, for example, may be in a range of X^{a} to Y^{b}, wherein X^{a} is any of 1.3, 1.4, 1.5, 1.6, 1.7, 1.8, 1.9, 2.0, 2.1, 2.2, 2.3 or 2.4 and Y^{b} is any of 2.1, 2.2, 2.3, 2.4, 2.5, 2.6, 2.7, 2.8, 2.9, or 3.0 and X^{a}<Y^{b}. For example, the ratio of the k_{cat}/K_{M} of a variant to control TEV protease may be in a range of 1.3-3.0 or 1.4-2.9 or 1.5-2.8 or 1.6-2.7.

In some embodiments, 1 unit of a variant TEV protease may cleave 2 μg of MBP-fusion protein, MBP5-TEV-paramyosin ΔSal, to 95% completion in a total reaction volume of 10 μl in 1 hour at 30° C. in 50 mM Tris-HCl (pH 7.5 @ 25° C.) with 0.5 mM EDTA and 1 mM DTT.

All publications, patents, and patent applications mentioned in this specification are herein incorporated by reference to the same extent as if each individual publication, patent, or patent application was specifically and individually indicated to be incorporated by reference. Reagents referenced in this disclosure may be made using available materials and techniques, obtained from the indicated source, and/or obtained from New England Biolabs, Inc. (Ipswich, MA).

### Enzymes and Compositions

The present disclosure relates, in some embodiments, to a means for cleaving a protein of interest at cold temperatures (e.g., ≤20° C., ≤18° C., ≤16° C., ≤15° C., ≤14° C., ≤12° C., ≤10° C., ≤8° C., ≤6° C., ≤5° C., ≤4° C., ≤3° C., and/or ≤2° C.). Examples of a means for cleaving a protein of interest at cold temperatures include all of the variant TEV proteases having the physical and chemical properties disclosed herein (e.g., sequences, motifs, bonds, binding properties, and other structures and features). In some embodiments, the present disclosure relates to variant TEV proteases having one or more desirable properties including, for example, efficient, selective, and cold-active cleavage of one or more proteins of interest relative to, for example, wildtype TEV protease.

The present disclosure relates, in some embodiments, to an immobilized enzyme comprising a support and an enzyme immobilized thereto. For example, an immobilized variant TEV protease may comprise a variant TEV protease, a glycine-serine linker attached to the variant TEV protease by a peptide bond, a protein tag (e.g., a SNAP-tag) attached to the linker by a peptide bond, O⁶-benzylguanine bound to the protein tag (e.g., SNAP-Tag®); and beads (e.g., magnetic beads) having a surface modification comprising the O⁶-benzylguanine. In some embodiments, a support of an immobilized variant TEV protease may comprise a magnetic bead. A magnetic bead may comprise, for example, one or more surface modifications. Surface modifications may include, for example, O⁶-benzylguanine and/or PEG750. In some embodiments, an immobilized enzyme may comprise a ligand (e.g., O⁶-benzylguanine) and a receptor or tag (e.g., a SNAP-Tag®) capable of binding the ligand. For example, ligands may be disposed on a support and corresponding receptors may be disposed on (e.g., covalently attached to) an enzyme to be immobilized on the support. An immobilized enzyme may comprise, in some embodiments, an enzyme (e.g., variant TEV protease), optionally, a first linker (e.g., a peptide linker) attached to the enzyme, a polypeptide tag (e.g., a SNAP-Tag®) attached to the first linker, if present, or the enzyme, a ligand corresponding to the polypeptide tag (e.g., O⁶-benzylguanine) attached (e.g., covalently attached) to the tag, optionally, a second linker (e.g., polyethylene glycol) attached to the ligand, and a support (e.g., a magnetic bead) attached to the second linker if present or the ligand, the structure of which may be illustrated, in an N->C direction, as:

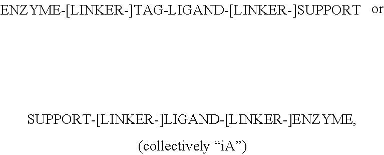

wherein dashes represent bonds (covalent or non-covalent) and brackets represent optional elements.

According to some embodiments, a variant TEV protease composition may comprise a variant TEV protease and, optionally, any of (including one or more of) a buffering agent (e.g., a storage buffer, a reaction buffer), an excipient, a salt (e.g., NaCl, MgCl₂, CaCl₂)), a protein (e.g., an internal control with or without a TEV protease recognition sequence), a stabilizer, a detergent (for example, ionic, non-ionic, and/or zwitterionic detergents (e.g., octoxinol, polysorbate 20), a polynucleotide, a cell (e.g., intact, digested, or any cell-free extract), a biological fluid or secretion (e.g., mucus, pus), an aptamer, a pH indicator (e.g., azolitimin, bromocresol purple, bromothymol blue, methylene blue, cresol red, neutral red, naphtholphthalein, phenol red), a crowding agent, a sugar (e.g., a mono, di, tri, tetra, or higher saccharide), a starch, cellulose, a glass-forming agent (e.g., glycerol, raffinose, stachyose, or trehalose for lyophilization), a lipid, an oil, aqueous media, a support (e.g., a bead) and/or (non-naturally occurring) combinations thereof. Combinations may include for example, two or more of the listed components (e.g., a salt and a buffer) or a plurality of species of a single listed component (e.g., two different salts or two different sugars). According to some embodiments, variant TEV protease compositions may comprise (a) a variant TEV protease, and (b) a protein (e.g., a known variant TEV protease substrate, a candidate variant TEV protease substrate, a test sample comprising or potentially comprising a variant TEV protease substrate), or a cellular extract or a cell-free preparation comprising a variant TEV protease substrate).

A variant TEV protease composition may comprise, for example, a variant TEV protease (e.g., having an amino acid sequence at least 85% identical to one or more of SEQ ID NOS: 1-28) and having at least one substitution relative to wildtype TEV protease. A variant TEV protease composition may be free of one or more other catalytic activities. For example, a variant TEV protease may be free of other proteases (e.g., non-specific proteases or proteases having other cleavage recognition sites), free of nucleases (e.g., RNases and/or DNases), free of polymerases activity, free of RNA and/or DNA modification activity, free of kinase activity, and/or free of phosphorylation and/or glycosylation activities, in each case, under desired test conditions (e.g., conditions of time, temperature, pH, salinity, model or intended substrate and/or others), for example, conditions intended to replicate conditions of a specific use of the variant TEV protease composition or intended to represent conditions for a range of uses.

In some embodiments, variant TEV proteases and compositions comprising one or more variant TEV proteases may have any desirable form including, for example, a liquid, a gel, a film, a powder, a cake, and/or any dried or lyophilized form. A variant TEV protease composition may comprise a variant TEV protease and a support or matrix, for example, a film, gel, fabric, column or bead comprising, for example, a magnetic material, agarose, polystyrene, polyacrylamide, and/or chitin. A variant TEV protease and compositions comprising a variant TEV protease may be active at higher temperatures. For example, a variant TEV protease or a variant TEV protease composition may display variant TEV protease activity at 25° C.-50° C. In some embodiments, a variant TEV protease may be cold-active and/or psychrophilic. For example, a variant TEV protease or a variant TEV protease composition may display variant TEV protease activity at temperatures below 20° C., below 18° C., below 16° C., below 14° C., below 12° C., below 10° C., below 8° C., below 6° C., below 4° C., below 2° C. or below 0° C. A composition may have a fluid state at temperatures near or below 0° C., for example, where the composition has a melting temperature below the desired temperature. Aqueous compositions may include, for example, one or more elements that reduce the composition's melting temperature including, for example, DMSO, methanol, glycerol, ethylene glycol, propylene glycol, sugars, amino acids, and proteins among others.

In some embodiments, a variant TEV protease may be encoded by a nucleic acid sequence that, when transcribed, translated, and/or processed, results in an amino acid sequence having at least 75%, at least 80%, at least 85%, at least 88%, at least 90%, at least 91%, at least 93%, at least 95%, at least 96%, at least 97%, at least 98% or at least 99% identity to SEQ ID NO:1. In some embodiments, a variant TEV protease may be encoded by a nucleic acid sequence that, when transcribed, translated, and/or processed, results in an amino acid sequence having at least 75%, at least 80%, at least 85%, at least 88%, at least 90%, at least 91%, at least 93%, at least 95%, at least 96%, at least 97%, at least 98% or at least 99% identity to any of SEQ ID NOS: 1-30 (e.g., 1-28) and including one or more substitutions relative to SEQ ID NO:31. A nucleic acid encoding a variant TEV protease may be included in an expression cassette, expression vector, or other expressible form suitable for in vitro or in vivo expression (e.g., in E. coli or other bacteria or P. pastoris or other yeast). A nucleic acid encoding a variant TEV protease may be modified or optimized (e.g., codon optimized) for expression in a desired organism or cell-free expression system.

### Methods and Workflows

Variant TEV proteases disclosed herein may be useful in many methods and/or workflows including manufacturing, molecular, cellular, research, sequencing, screening, diagnostic, and/or therapeutic applications. For example, variant TEV proteases may be used in any application where it is desirable to cleave a molecule (e.g., a protein) comprising a TEV protease recognition sequence (e.g., SEQ ID NO:34). Variant TEV proteases may cleave one or more polypeptides comprising a TEV protease recognition sequence (e.g., SEQ ID NO: 34) with greater efficiency and/or at lower temperatures than, for example, wildtype TEV protease. A molecule (e.g., a protein) for cleavage by a variant TEV protease may comprise a protein of interest, a removable segment, and a TEV protease recognition sequence disposed between the protein of interest and the removable segment. A molecule for cleavage may further comprise a first linker disposed between the protein of interest and the TEV protease recognition sequence and/or a second linker disposed between the TEV protease recognition sequence and the removable segment, wherein the first and second linker (if both present) may be the same or different. An example of a molecule for cleavage may be illustrated, in an N->C direction, as:

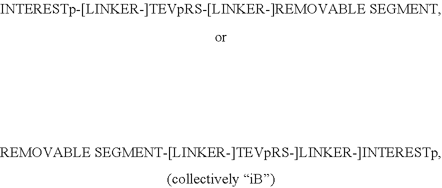

wherein dashes represent bonds (covalent or non-covalent) and brackets represent optional elements. A protein of interest may be at or closer to the N-terminal end than the removable segment in some embodiments. A protein of interest, according to some embodiments, may be at or closer to the C-terminal end than the removable segment. A protein of interest may be or comprise one or more of a receptor, a ligand, an enzyme, an effector, an antibody, a nucleic acid binding protein, a structural protein, a hormone, a contractile protein, a storage protein, a transport protein, a chaperone, a histone, a lipoprotein, a glycoprotein, a toxin, an amyloid, a variant protein, a misfolded protein (or any other structurally distinctive protein), and/or a protein bound (covalently or non-covalently) to another molecule (e.g., a protein: RNA complex, a protein: DNA complex). A removeable segment may be or comprise one or more of a purification tag, an affinity tag, a fluorophore, a solubility tag, an epitope tag, an expression enhancing tag, and/or a second protein of interest.

A protein of interest and/or a fusion protein comprising the protein of interest may have one or more desirable properties (e.g., lower catalytic activity, increased stability, improved binding to desired molecule, reduced binding to an unwanted molecule) that are improved at lower temperatures (e.g., temperatures below 20° C., below 18° C., below 16° C., below 14° C., below 12° C., below 10° C., below 8° C., below 6° C., below 4° C., below 2° C. or below 0° C.). Accordingly, it may be desirable to limit or control conditions to which a protein of interest and/or a fusion protein is exposed to preserve the display of one or more cold-enhanced property. Provided variant TEV proteases may be used in methods and workflows to perform a desired or required cleavage while avoiding undesirable conditions (e.g., higher temperatures, longer incubation times, higher protease concentrations) that would obtain if using a reference protease (e.g., a wildtype TEV protease).

A method may comprise, in some embodiments, (a) contacting (i) a TEV protease substrate comprising a TEVpRS, a protein of interest attached (e.g., covalently) to the TEVpRS, and a removable segment attached (e.g., covalently) to the TEV protease recognition sequence (e.g., according to iB), and (ii) a variant TEV protease (e.g., a soluble variant TEV protease or an immobilized (e.g., according to iA) variant TEV protease) to form a cleavage product mixture comprising separately the protein of interest and the removable segment and (b) optionally separating the protein of interest from the removable segment. Separating the protein of interest may be performed by any desired technique including, for example, affinity fractionation, size fractionation, capillary electrophoresis, filtration, and/or dialysis. Variant TEV proteases may allow methods to be performed at lower temperatures, for example, where a protein of interest is more stable or otherwise has a desirable structure or other property. For example, the (a) contacting and/or the (b) separating each independently may be performed at a temperature below 20° C., below 18° C., below 16° C., below 14° C., below 12° C., below 10° C., below 8° C., below 6° C., below 4° C., below 2° C. or below 0° C.

In some embodiments, variant TEV proteases may be used in applications involving proteins comprising a TEVpRS. For example, a protein of interest may naturally comprise or may be engineered to comprise a TEVpRS. Methods of using a variant TEV protease may include contacting a variant TEV protease with a protein of interest comprising a TEVpRS (e.g., at low temperature) to form cleavage products (e.g., cleavage products that are free of the intact protein of interest). It may be desirable to inactivate an enzyme having a TEVpRS, for example, upon completion of reaction(s) catalyzed by the enzyme or to prevent the enzyme from becoming active in the presence of a potential substrate. In some embodiments, a method may include contacting an enzyme having a TEVpRS with a substrate at a first temperature (e.g., above 15° C., above 18° C., above 20° C., above 30° C.) to produce reaction products, contacting the reaction products (or a fraction thereof comprising the enzyme) with a variant TEV protease at a second temperature (e.g., below 10° C., below 5° C.) to produce enzyme cleavage products, wherein the enzyme cleavage products optionally (i) are free of the intact protein having a TEVpRS and/or (ii) comprise at least two fragments of the protein having a TEVpRS. In some embodiments, the first and second temperature may optionally be the same. In some embodiments, both the enzyme-catalyzed reaction of interest and the enzyme inactivation reactions may be performed in a single container (“one pot”).

A method, according to some embodiments, may comprise contacting (a) a composition (e.g., a patient specimen, a cell fraction, a cell extract, or a manufacturing intermediate) comprising or potentially comprising a protein (e.g., a target protein) having a TEVpRS with (b) a variant TEV protease (e.g., a soluble variant TEV protease or an immobilized (e.g., according to iA) variant TEV protease) to produce a cleavage product mixture, wherein the cleavage product mixture optionally (i) is free of the intact protein having a TEVpRS and/or (ii) comprises at least two fragments of the protein having a TEVpRS, wherein the contacting is performed at a temperature below 20° C., below 18° C., below 16° C., below 14° C., below 12° C., below 10° C., below 8° C., below 6° C., below 4° C., below 2° C. or below 0° C.

In some embodiments, enzyme cleavage products and/or a cleavage product mixture may comprise little or none of the intact protein (e.g., a target protein) having a TEVpRS. For example, enzyme cleavage products and/or a cleavage product mixture may comprise ≤5 mol. %, ≤4 mol. %, ≤3 mol. %, ≤2 mol. %, ≤1 mol. %, or ≤0.5 mol. % of the initial concentration of the intact protein (e.g., a target protein) having a TEVpRS. In some embodiments, ≥95 mol. %, ≥96 mol. %, ≥97 mol. %, ≥98 mol. %, ≥99 mol. %, ≥99.5 mol. %, or ≥99.9 mol. % of a protein comprising a TEVpRS substrate may be cleaved by contact with a variant TEV protease, for example, at temperatures below 20° C., below 18° C., below 16° C., below 14° C., below 12° C., below 10° C., below 8° C., below 6° C., below 4° C., below 2° C. or below 0° C.

The present disclosure relates to methods for detecting molecular interactions. For example, temperature sensitive interactions (e.g., protein: protein interactions, enzyme: substrate, enzyme: cofactor, protein: polynucleotide interactions) may occur, may have higher affinity, and/or may have higher avidity at lower temperatures (e.g., temperatures below 20° C., below 18° C., below 16° C., below 14° C., below 12° C., below 10° C., below 8° C., below 6° C., below 4° C., below 2° C. or below 0° C. Detection systems that rely on cleavage (e.g., proteolytic cleavage) at higher temperatures may not have the capacity to detect such temperature sensitive interactions (e.g., because the cleavage does not occur at all or does not occur as efficiently as needed to support detection).

In some embodiments, a TEV protease substrate may comprise a TEV protease recognition sequence (“TEVpRS”), a support (e.g., a matrix or a bead) attached (e.g., covalently) to the TEV protease recognition sequence (e.g., with or without a linker), and a bait molecule (e.g., a protein receptor, a ligand, an enzyme, an effector, an antibody, a nucleic acid binding protein) attached (e.g., covalently) to the TEV protease recognition sequence (e.g., with or without a linker). A TEV protease substrate may be illustrated, in an N->C direction, as:

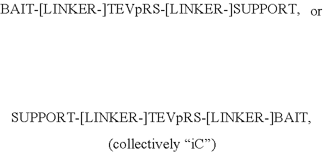

wherein dashes represent bonds (covalent or non-covalent) and brackets represent optional elements.

A method may comprise (a) contacting (i) a TEV protease substrate comprising a TEVpRS, a support attached (e.g., covalently) to the TEV protease recognition sequence, and a bait molecule attached (e.g., covalently) to the TEV protease recognition sequence (e.g., according to iC) and (ii) a composition comprising (or optionally a composition potentially comprising) a target molecule capable of binding the bait molecule (e.g., with high affinity and/or specificity) to form one or more TEV protease substrate: target molecule complexes (e.g., comprising one or more bait: target pairings), and (b) contacting the one or more TEV protease substrate: target molecule complexes with a variant TEV protease to cleave the TEV protease substrate at the recognition sequence. In some embodiments, cleaving the TEV protease substrate releases the bait: target pairings from the support. Methods may further comprise analyzing released bait: target pairings. For example, a method may include analyzing the target member, the bait member, or both members of one or more bait: target pairings. Variant TEV proteases may allow methods to be performed at lower temperatures, for example, where bait: target pairings are more stable, more specific, or otherwise improved. For example, (a) contacting and/or (b) contacting of a method may be performed at temperatures below 20° C., below 18° C., below 16° C., below 14° C., below 12° C., below 10° C., below 8° C., below 6° C., below 4° C., below 2° C. or below 0° C. In some embodiments, (a) contacting may be performed at a first temperature (e.g., above 15° C., above 18° C., above 20° C., above 30° C.) and (b) contacting may be performed at a second temperature, wherein the second temperature is lower than the first temperature (e.g., below 10° C., below 5° C.).

In some embodiments, ≥95 mol. %, ≥96 mol. %, ≥97 mol. %, ≥98 mol. %, ≥99 mol. %, ≥99.5 mol. %, or ≥99.9 mol. % of the TEV protease substrate: target molecule complexes are cleaved at the recognition sequence by contact with a variant TEV protease, for example, at temperatures below 20° C., below 18° C., below 16° C., below 14° C., below 12° C., below 10° C., below 8° C., below 6° C., below 4° C., below 2° C. or below 0° C. In some embodiments, a variant TEV protease may cleave a TEV protease substrate: target molecule complex with a higher catalytic efficiency than a control TEV protease. For example, for a given substrate (e.g., a TEV protease substrate: target molecule) and at a given temperature (e.g., 4° C.), the ratio of the k_{cat}/K_{M} of a variant TEV protease to the k_{cat}/K_{M} of a control TEV protease, for example, may be in a range of X^{a} to Y^{b}, wherein X^{a} is any of 1.3, 1.4, 1.5, 1.6, 1.7, 1.8, 1.9, 2.0, 2.1, 2.2, 2.3 or 2.4 and Y^{b} is any of 2.1, 2.2, 2.3, 2.4, 2.5, 2.6, 2.7, 2.8, 2.9, or 3.0 and X^{a}<Y^{b}. For example, the ratio of the k_{cat}/K_{M} of a variant to control TEV protease may be in a range of 1.3-3.0 or 1.4-2.9 or 1.5-2.8 or 1.6-2.7.

### Kits

The present disclosure further relates to kits including a variant TEV protease. For example, a kit may include a variant TEV protease, other proteins (e.g., positive and/or negative control substrates), other enzymes, buffering agents, or combinations thereof. Enzymes may be included in a storage buffer (e.g., comprising glycerol and a buffering agent). A kit may include a reaction buffer which may be in concentrated form, and the buffer may contain additives (e.g. glycerol), salt (e.g. KCl), reducing agent, EDTA or detergents, among others. A kit may be a non-natural collection of components configured, for example, for convenient storage, shipping, delivery, and/or use. One or more components of a kit may be included in one container for a single step reaction, or one or more components may be contained in one container, but separated from other components for sequential use or parallel use. The contents of a kit may be formulated for use in a desired method or process.

A kit is provided that contains: (i) a variant TEV protease; and (ii) a buffer. A variant TEV protease may have any desired form including, for example, a liquid form, a gel form, an aqueous form, a film form, a crystalline form, a powder form, a cake form, a dried form, freeze dried form, a lyophilized form, and an immobilized form. A variant TEV protease may be included with or in a buffer (e.g., a storage buffer or a reaction buffer in concentrated form). A kit may contain a variant TEV protease in a mastermix suitable for receiving and amplifying a template nucleic acid of interest. A variant TEV protease may be a purified enzyme so as to contain substantially no DNA, no RNA, no nucleases, and/or no other proteases. The reaction buffer in (ii) and/or storage buffers containing or included with the TEV protease in (i) may include non-ionic, ionic (e.g. anionic or zwitterionic) surfactants and/or crowding agents. A kit may include a variant TEV protease and the reaction buffer in a single container or in different containers.

A kit may further include instructions for using the components of the kit to practice a desired method. The instructions may be recorded on a suitable recording medium. For example, instructions may be printed on a substrate, such as paper or plastic, etc. As such, the instructions may be present in the kits as a package insert, in the labeling of the container of the kit or components thereof (i.e., associated with the packaging or sub-packaging) etc. Instructions may be present as an electronic storage data file residing on a suitable computer readable storage medium (e.g. a CD-ROM, a flash drive). Instructions may be provided remotely using, for example, cloud or internet resources with a link or other access instructions provided in or with a kit.

### EXAMPLES

Some specific example embodiments may be illustrated by one or more of the examples provided herein.

### Example 1: Synthesis of TEV Protease Variants

A codon-optimized TEV protease gene with L56V, S135G and S219V and an N-terminal His tag (SEQ ID NO:30) was cloned into pUCT7 plasmid, which is a pUC19 based plasmid with T7 promoter followed by Shine-Dalgarno sequence upstream of open reading frame (ORF), and T7 terminator sequence downstream of ORF. L56V and S135G substitutions were included to improve solubility, and S219V was included to prevent autoproteolysis. The TEV protease with these three substitutions (the “TEV protease triple variant” or “control”) was used as a reference herein. Additional substitutions added to the TEV protease triple variant sequence included one or more of K45A, K45G, K45H, K45Q, K45R, K45S, R49E, R49K, R49V, I77C, I77L, I77V, D78E, D78G, D78K, D78Q, M82A, M82I, M82L, M82V, I83L, 183M, I83V, I84L, 184V, Q150D, Q150F, Q150H, S153L, S153N, L155A, L155I, L155M and L155V, wherein substitutions are numbered according to amino acid positions of wildtype TEV protease (SEQ ID NO:31).

Plasmids harboring TEV protease variants were constructed using Q5 Site-Directed Mutagenesis Kit (New England Biolabs). A modified version of PURExpress (New England Biolabs) was used to synthesize TEV protease variants in vitro. 100 to 200 ng of plasmid and 20 Units of RNase Inhibitor, Murine (New England Biolabs) was added in 25 μl of the modified PURExpress reaction and the reaction was incubated at 37° C. for 3 hours. Reactions were stored at −20° C. pending further analysis.

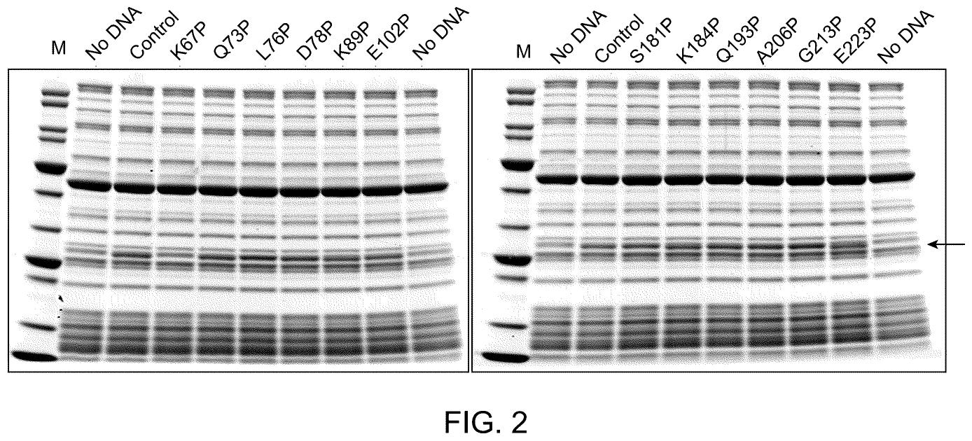

Alternatively, N-terminal histidine tagged TEV protease variants were expressed in vivo as maltose-binding protein fusions. A TEV protease cleavage site positioned between the MBP and variant sequences allows intracellular cleavage of the fusion proteins. Cells were lysed and cleaved N-terminal, His-tagged TEV protease variants were purified chromatographically. Transformed T7 Express competent cells were grown in 1 L of LB media at 37° C. until OD is 0.8 A₆₀₀. Then 1 mL of 0.5 M IPTG was added, and the culture was incubated at 18° C. overnight. The cells were harvested and suspended in 50 mL of a Tris-buffered solution (pH 7.5). The suspended cells were lysed using Microfluidizer (Dyhydromatics) and centrifuged at 15,000 rpm for 20 min twice to remove cell debris. The supernatant was loaded on HisTrap FF 5 ml (GE Healthcare) and TEV protease was eluted with a gradient of a Tris-buffered solution (pH 7.5) comprising imidazole. The eluted protein was collected and dialyzed against a storage buffer comprising a reducing agent and 50% Glycerol at 4° C. overnight. The concentration of protein was determined by Bradford assay. Purified proteins were stored at −80° C. Example results are shown in FIG. 2.

### Example 2: Quantification of TEV Protease Variants in the In Vitro Synthesis Reactions

2.5 μl of the in vitro synthesis reaction was mixed with 7.5 μl of H₂O and 5 μl of 3×SDS Sample Buffer (New England Biolabs). The solution was incubated at 95° C. for 5 min, spun at 14,000 rpm for 1 min, and loaded on Novex™ WedgeWell™ 4-20% Tris-Glycine gels (Invitrogen). The reaction without plasmid was loaded as a negative control. Unstained Protein Standard, Broad Range (10-200 kDa) (New England Biolabs) was used as a marker. The gel was stained using SimplyBlue™ SafeStain (Life Technologies) and the band of TEV protease variant was quantified using Odyssey Imaging System (LI-COR Biosciences). Then the concentration of TEV protease variant relative to control TEV protease was calculated.

### Example 3: Fluorogenic Peptide Cleavage Assays for TEV Protease Variants

A fluorogenic peptide (5-FAM-ENLYQGIV-K (QXL520)-NH2; SEQ ID NO:32) was used as a substrate (AnaSpec). It is 9 amino acids long, which includes a TEV protease recognition sequence (SEQ ID NO:34). A fluorophore, 5-FAM, is attached to the N-terminal, and a quencher, QXL520, is attached to the epsilon amino group of C-terminal lysine residue. If the peptide is cleaved by TEV protease, it shows fluorescence. Excitation and emission wavelengths are 493 nm and 517 nm, respectively.

2.5 μl of 10×TEV Protease Reaction Buffer (500 mM Tris-HCl, pH 7.5, 5 mM EDTA, and 10 mM DTT) (New England Biolabs), 2.5 μl of 100 μM peptide substrate and 19 μl of H₂O were mixed in a tube. Reactions were started by adding 1 μl of in vitro synthesis reaction diluted with H₂O. Each reaction was incubated at 15° C. for 30 min and then heat-inactivated at 65° C. for 10 min using T100 Thermal Cycler (Bio-Rad). Aliquots of inactivated reaction mixtures were added to wells of a 384-well plate (10 μL mixture/well), and fluorescence was measured using SpectraMax M5 (Molecular Devices). Measurements were performed 4 times and the average values were calculated.

### Example 4: CBD-TEVrs-bglA Cleavage Assay

As a substrate of purified TEV protease variants, CBD-TEVrs-bglA was prepared. This protein is composed of N-terminal His-tagged chitin binding domain (CBD) followed by TEVrs (ENLYFQG) and 6-phospho-β-glucosidase A (bglA) (SEQ ID NO:33). The CBD-TEVrs-bglA gene was cloned into pET28 vector. CBD-TEVrs-bglA was expressed in vivo and purified with nickel columns in the same way as TEV protease variants described above.

5 μl of 10×TEV Protease Reaction Buffer, 5 μl of 370 μM CBD-TEVrs-bglA, and 38 μl of H₂O were mixed in a tube. Cleavage reactions were started by adding 2 μl of 10 μM TEV protease in the tube and reaction tubes were incubated at the selected temperature for the selected time (0° C. for 18 hours, 4° C. for 18 hours, 20° C. for 3 hours or 30° C. for 2 hours) using a thermocycler with aliquots (5 μl each) removed at intervals (every 30 minutes for the reactions at 20° C. and every 20 minutes for the reactions at 30° C.), mixed with cold acetone (50 μl), and kept on ice until all intervals for a given treatment were collected.

Tubes were spun at 14,000 rpm for 10 min at 4° C. Then supernatant was discarded, and pellet was dried at room temperature for 10 min. The pellet was dissolved in 40 μl of H₂O and 20 μl of 3×SDS Sample Buffer (New England Biolabs). Solutions in the tubes were incubated at 95° C. for 5 min and spun at 14,000 rpm for 1 min, after which 10 μl of solution (2 μg of CBD-TEVrs-bglA) was loaded on Novex™ WedgeWell™ 4-20% Tris-Glycine gel (Invitrogen). The gel was stained using SimplyBlue™ SafeStain (Life Technologies) and the bands of CBD-TEVrs-bglA and bglA were quantified using Odyssey Imaging System (LI-COR Biosciences), from which the cleavage ratio of CBD-TEVrs-bglA was calculated.

### Example 5: Titration with Control and TEV Protease Variants

Stock solutions (50 μM) of control or TEV protease variants were diluted with storage buffer (50 mM Tris-HCl, pH 7.5, 250 mM NaCl, 1 mM DTT, 50% Glycerol) in 2-fold serial order. 5 μl of 10×TEV Protease Reaction Buffer, 5 μl of 370 μM CBD-TEVrs-bglA, and 38 μl of H₂O were mixed in a tube. Cleavage reaction started by adding 2 μl of diluted TEV protease in the tube, and the reactions were incubated at 4° C. for 18 hours. Aliquots (5 μL) were taken from each tube and mixed with 50 μl of cold acetone. Tubes were spun at 14,000 rpm for 10 min at 4° C. Then supernatant was discarded, and pellet was dried at room temperature for 10 min. The pellet was dissolved in 40 μL of H₂O and 20 μL of 3×SDS Sample Buffer (New England Biolabs). The solutions in the tubes were incubated at 95° C. for 5 min, spun at 14,000 rpm for 1 min, and 10 μl of solution (2 μg of CBD-TEVrs-bglA) was loaded on Novex™ WedgeWell™ 4-20% Tris-Glycine gel (Invitrogen). The gel was stained using SimplyBlue™ SafeStain (Life Technologies) and the bands of CBD-TEVrs-bglA and bglA were quantified using Odyssey Imaging System (LI-COR Biosciences), from which the cleavage ratio of CBD-TEVrs-bglA was calculated.

### Example 6: Identifying Fluorogenic Peptide Cleavage Reaction Conditions

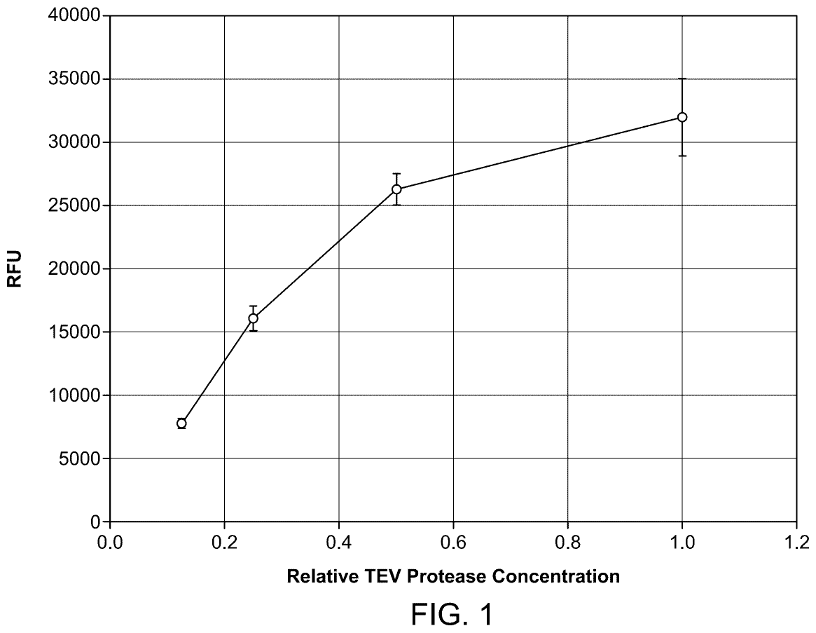

Products of in vitro synthesis reactions were diluted with water just prior to use in the fluorescence assay of Example 3 such that the intensity of fluorescence is proportional to the activity of TEV proteases. To determine the appropriate level of dilution, the correlation was measured between the concentration of control TEV protease and the fluorescence after 30 min incubation at 15° C. (FIG. 1). There was a good linear correlation when the control TEV protease is diluted by more than 4-fold. Therefore, the control TEV protease was diluted with water by 5-fold at the first round of screening. Since enzymatic activity depends on the concentration of enzyme, it is important to add the same amount of TEV protease variant as the control TEV protease in fluorogenic peptide cleavage reactions. The concentrations of TEV protease variants were diluted with water to match the concentration of control TEV protease diluted by 5-fold.

### Example 7: Analysis of Proline Variants Produced In Vitro

TEV protease triple variants further comprising a proline substitution, namely K67P, Q73P, L76P (SEQ ID NO:6), D78P, K89P (SEQ ID NO:7), E102P, S181P (SEQ ID NO:8), K184P, Q193P, A206P, G213P, or E223P (numbered according to SEQ ID NO:31), were synthesized in vitro as set forth in Example 1. In vitro synthesis products were assessed on SDS gels together with control TEV protease (SEQ ID NO:29) as set forth in Example 2. Results are shown in FIG. 2. Some variation in protein synthesis was noted among variants. TEV protease variant bands on SDS gels were quantified, and the concentrations of TEV protease variants were adjusted to match the concentration of control TEV protease.

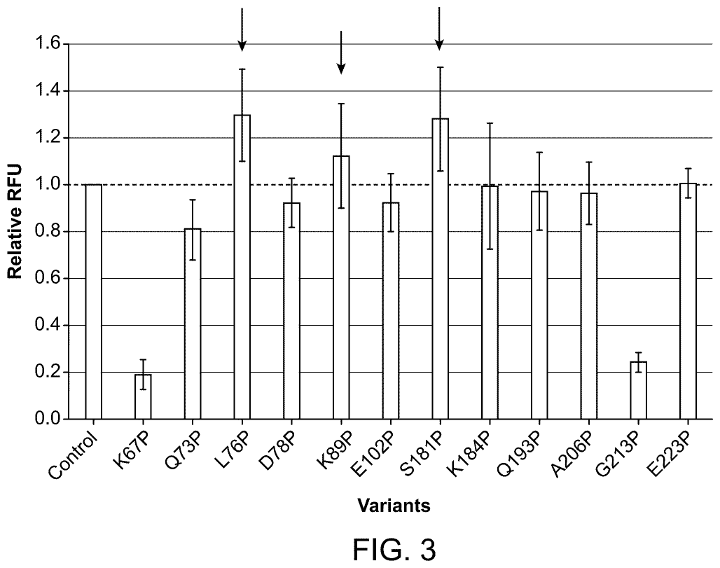

The cleavage reactions of fluorogenic peptide substrate with TEV protease variants were performed according to Example 3 at 15° C. for 30 minutes. Results are shown in FIG. 3. Of 12 TEV protease variants, L76P, K89P, and S181P variants showed higher activity than the control TEV protease (SEQ ID NO:29).

### Example 8: Analysis of Catalytic Triad Variants Produced In Vitro

TEV protease triple variants (SEQ ID NO:29) comprising a further substitution (K45A, K45G, K45H, K45Q, K45R, K45S, R49E, R49K, R49V, I77C, I77L, I77V, D78E, D78G, D78K, D78Q, M82A, M82I, M82L, M82V, I83L, 183M, I83V, 184L, 184V, Q150D, Q150F, Q150H, S153L, S153N, L155A, L155I, L155M or L155V with numbering according to SEQ ID NO:31) in or near TEV protease's predicted catalytic triad (H46, D81, and C151 with numbering according to SEQ ID NO:31) were synthesized in vitro as set forth in Example 1. Synthesis products were fractionated on SDS gels and TEV protease variant and control bands were quantified as set forth in Example 2. Concentrations of TEV protease variants were adjusted to match the concentration of control TEV protease.

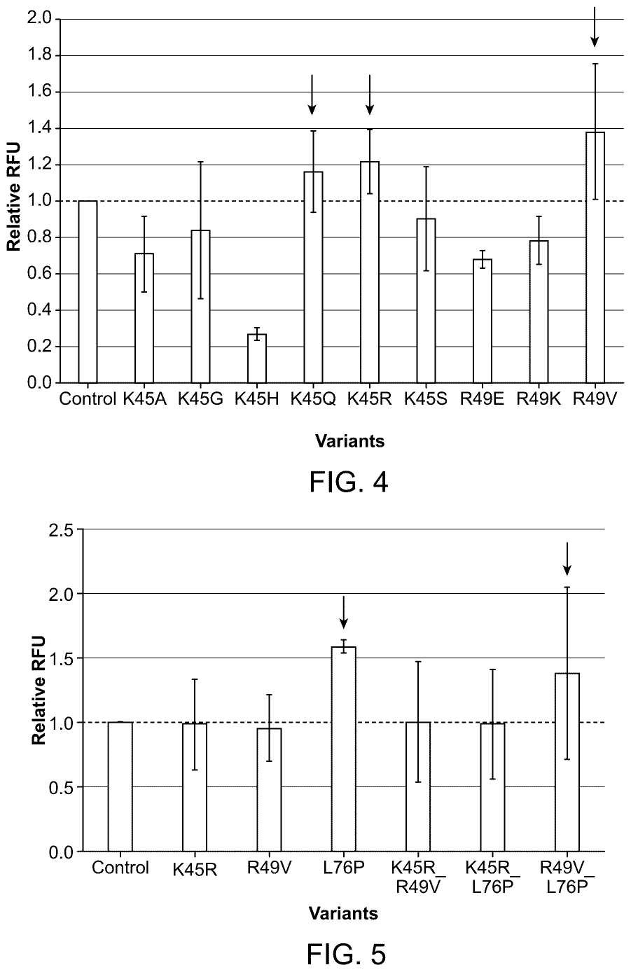

Nine substitutions were introduced at positions near H45 and assayed according to Example 3 at 15° C. for 30 minutes. Of these nine, K45Q, K45R, and R49V showed higher activity than control TEV protease (SEQ ID NO:29) (FIG. 4). Sixteen substitutions were introduced at positions near D81 and nine substitutions were introduced at positions near C151, some of which displayed catalytic activity, though none exceeded the activity of the control TEV protease under the conditions tested.

### Example 9: Analysis of Combined Variants Produced In Vitro

TEV protease triple variants (SEQ ID NO:29) comprising further substitutions K45R, R49V, L76P, K45R_R49V, K45R_L76P, or R49V_L76P (numbering according to SEQ ID NO: 31) were synthesized in vitro as set forth in Example 1, concentrations adjusted, and assayed at 15° C. for 30 minutes according to Example 3. Results are shown in FIG. 5. TEV protease triple variants further comprising K45R, R49V, K45R_R49V, or K45R_L76P each displayed activity comparable to the control. The variant with the R49V_L76P substitutions (SEQ ID NO:3) and the variant with the L76P substitution (SEQ ID NO:6) displayed activity beyond (~1.4×-~1.5×) the activity of the control.

### Example 10: Analysis of Variants Produced In Vivo

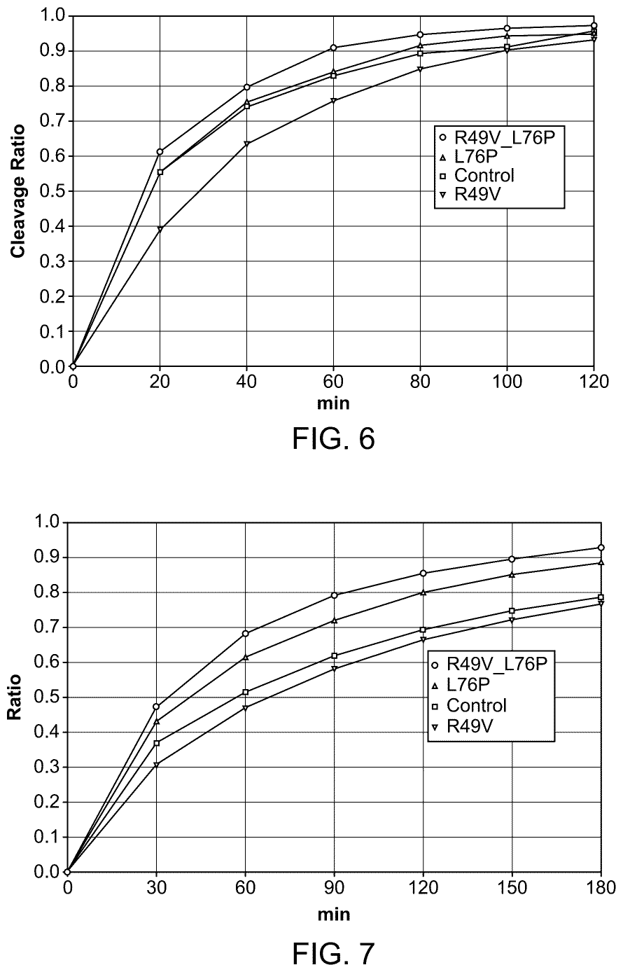

TEV protease variants of Example 9 were produced in vivo in accordance with Example 1. CBD-TEVrs-bglA cleavage reactions with the purified TEV protease variants were evaluated over time at 30° C. and 20° C. in accordance with Example 4. Results are shown in FIG. 6 (30° C.) and FIG. 7 (20° C.). The R49V variant showed slightly weaker activity than control TEV protease (SEQ ID NO:30). At both temperatures, the L76P variant showed higher activity than control TEV protease and the R49V_L76P variant showed higher activity than L76P variant.

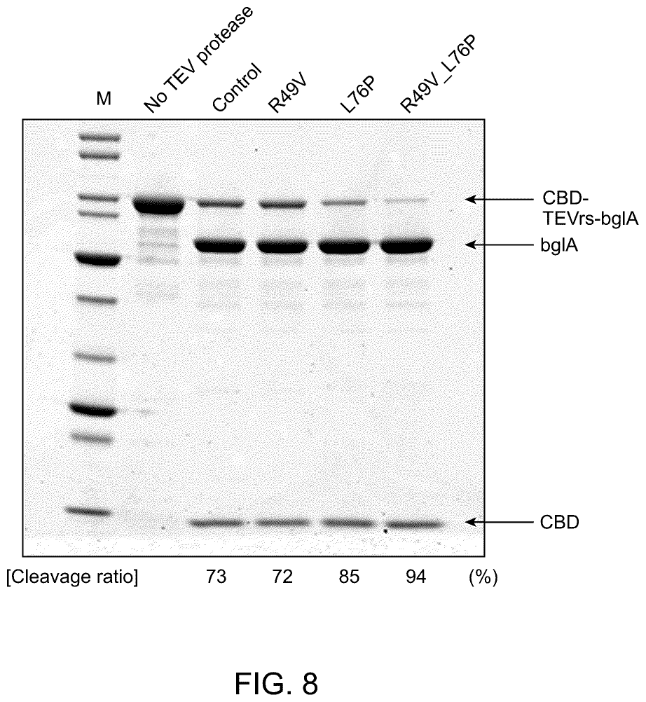

CBD-TEVrs-bglA cleavage with these TEV protease variants was assessed at 4° C. for 18 hours. Results are shown in FIG. 8. The difference in cleavage ratio among TEV protease variants was larger at 4° C. than 20° C. Control TEV protease cleaved the substrate by 73%, whereas R49V_L76P variant cleaved the substrate by 94%, indicating that the R49V_L76P variant is available to cleave protein substrates at 4° C. and does so with higher activity than control TEV protease or either variant containing only one of the R49V or L76P substitutions.

### Example 11: Analysis of Catalytic Triad Variants

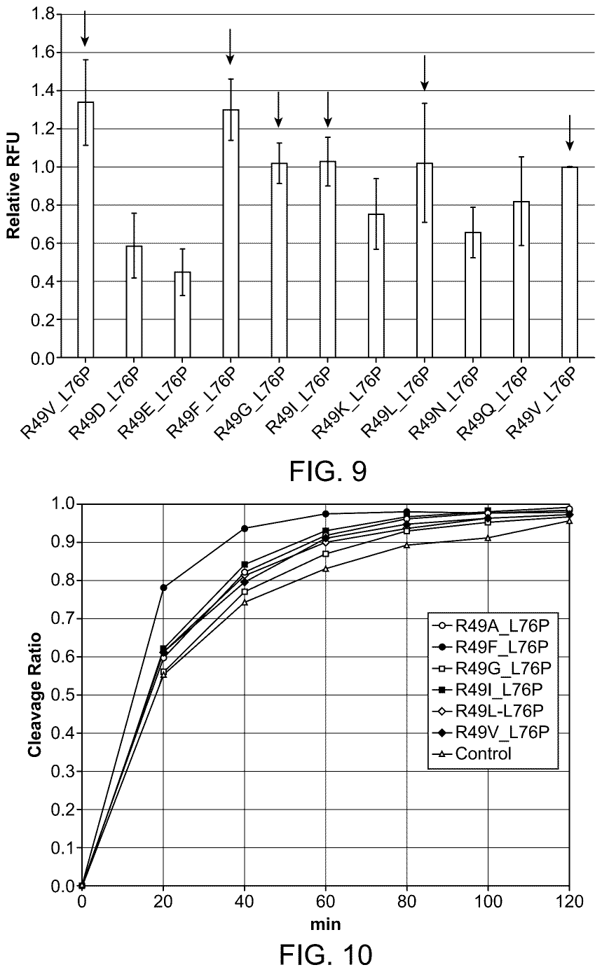

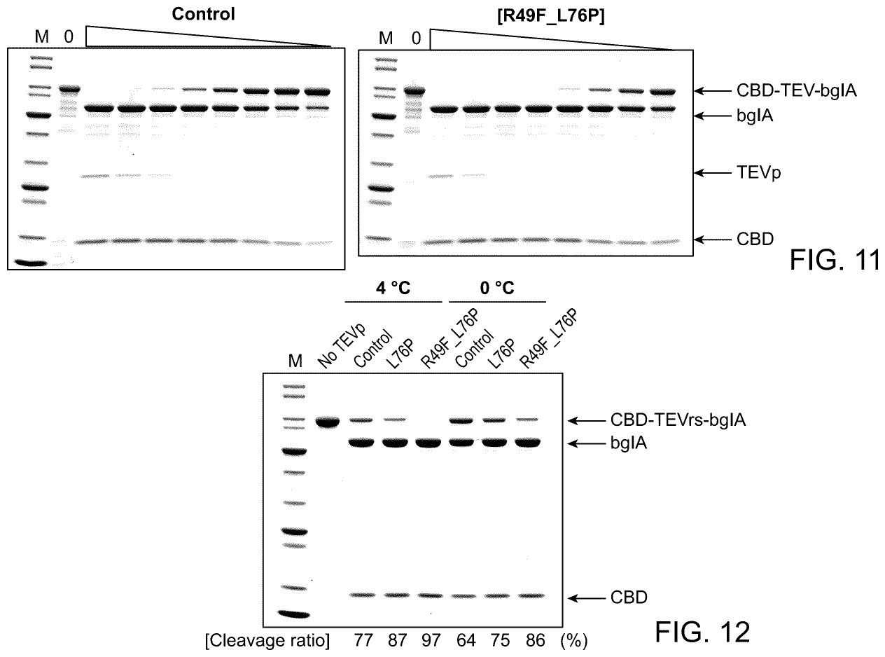

According to crystal structure data for TEV protease in complex with a substrate, amino acids R49 and L76 do not bind to the substrate, but they are positioned close to the catalytic triad. TEV protease triple variants further comprising one of eleven R49X_L76P substitutions were expressed in vitro as set forth in Example 1, concentrations adjusted, and evaluated in accordance with Example 3 at 10° C. for 60 minutes. As shown in FIG. 9, hydrophobic amino acid substitutions at R49 were associated with improved activity. Variants marked with arrows (R49A_L76P, R49F_L76P (SEQ ID NO:11), R49G_L76P, R49I L76P, R49L_L76P, R49V_L76P (SEQ ID NO: 12)) were expressed in vivo and evaluated in accordance with Example 4 at 30° C. As shown in FIG. 10, all variants performed better that the control with the R49F_L76P variant (SEQ ID NO: 11) displaying the highest activity.

### Example 12: Low Temperature Performance of Variant TEV Proteases

CBD-TEVrs-bglA cleavage by the L76P variant (SEQ ID NO: 15), the R49F_L76P variant (SEQ ID NO:11), the control variant protease (SEQ ID NO:30) was assessed at 0° C. or 4° C. for 18 hours according to Example 4. Results are shown in FIG. 12. Variants L76P and R49F_L76P were respectively ~10% and ~20% more active at both temperatures than control TEV protease. Under the 4° C. assay conditions, the control TEV protease cleaved 77% of the substrate whereas the L76P variant cleaved 87% of the substrate (10% more than the control) and the R49F_L76P variant cleaved 97% of the substrate (20% more than the control). Similarly, under the 0° C. assay conditions, the control TEV protease cleaved 64% of the substrate whereas the L76P variant cleaved 75% of the substrate (11% more than the control) and the R49F_L76P variant cleaved 86% of the substrate (22% more than the control).

### Example 13: Kinetic Analysis of Catalytic Triad Variant

The R49V variant (SEQ ID NO:14), the L76P variant (SEQ ID NO:15), the R49V_L76P variant (SEQ ID NO:12), and the R49F_L76P variant (SEQ ID NO:11) were selected for further analysis. CBD-TEVrs-bglA cleavage activity was measured as a function of TEV protease concentration (2-fold serial dilutions) at 4° C. for 18 hours according to Example 5.

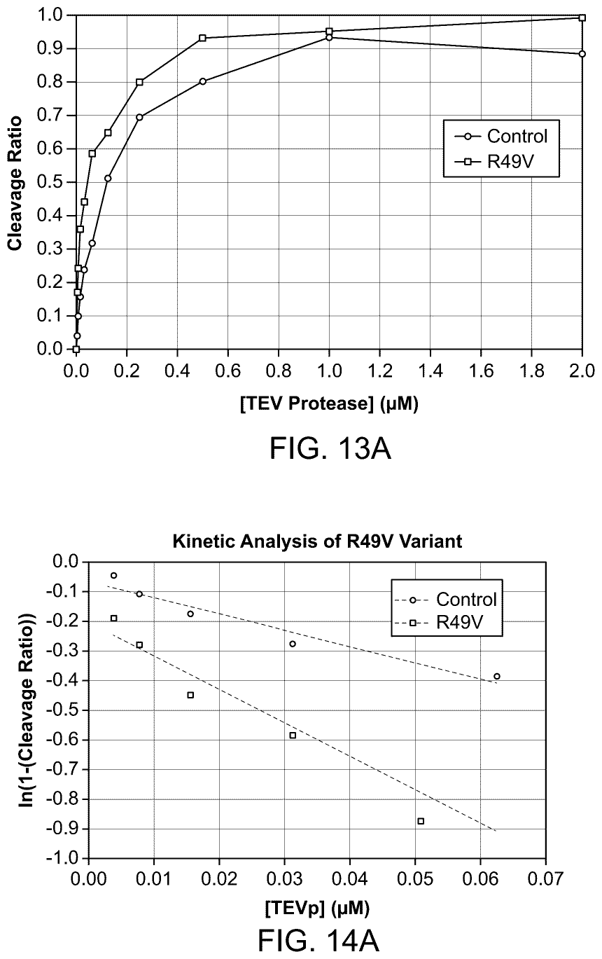

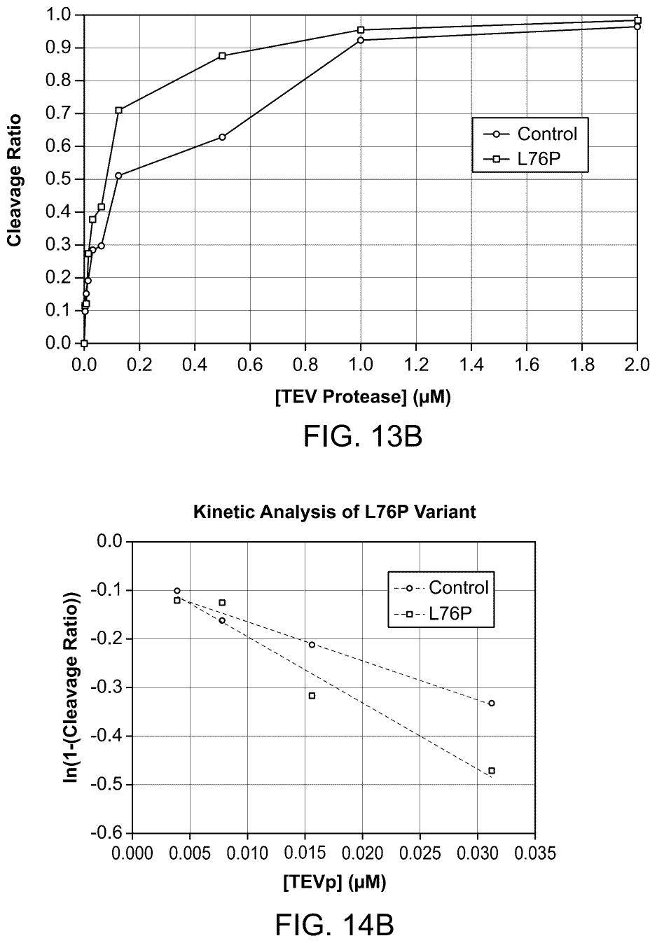

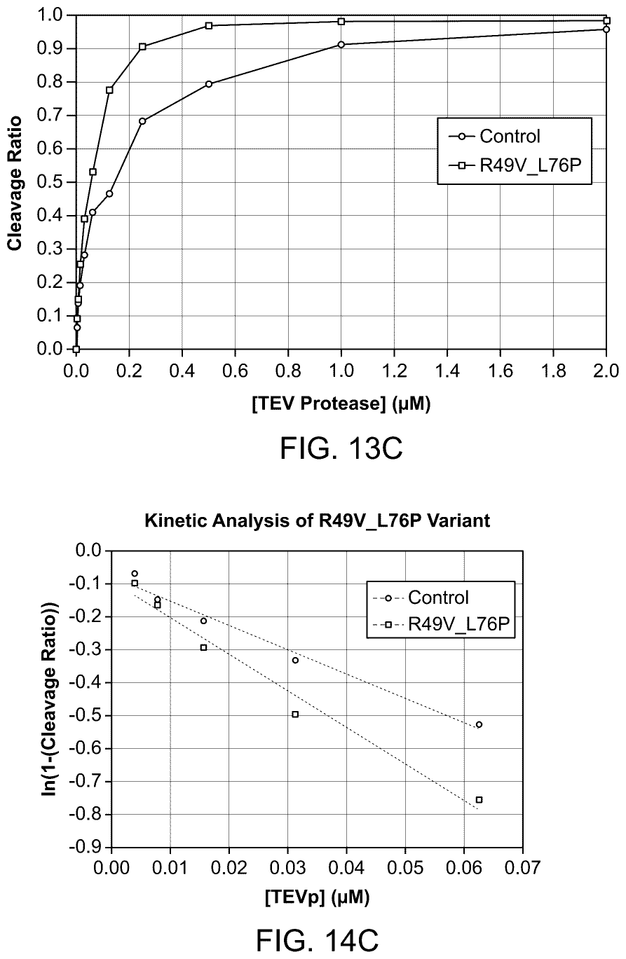

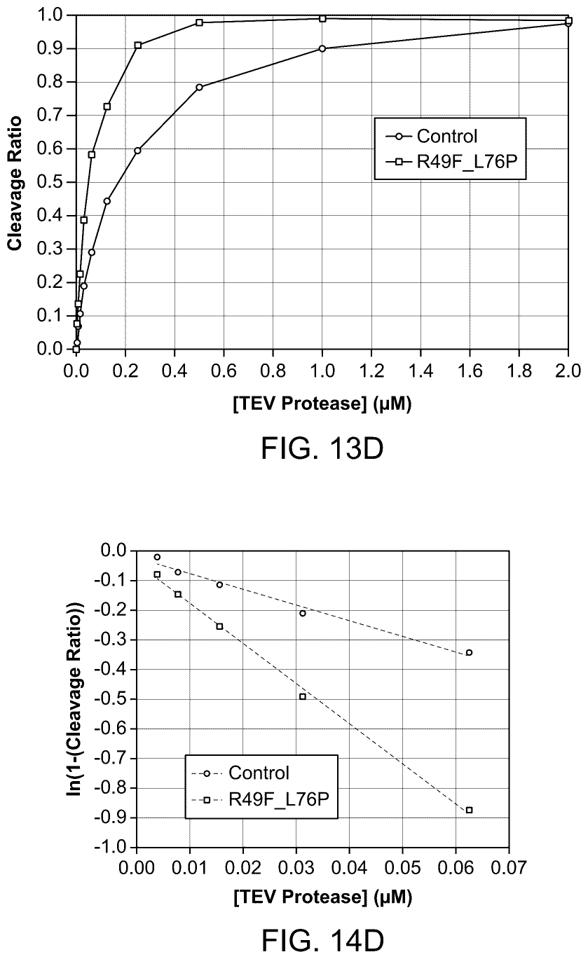

As shown in FIG. 13C, the R49V_L76P variant reached almost 100% cleavage at 0.5 μM, whereas 2 μM of the control TEV protease was needed to reach the same mark. As shown in FIG. 13D, the R49F_L76P variant reached almost 100% cleavage at 0.5 μM, whereas 2 μM of the control TEV protease was needed to reach the same mark.

Kinetic parameters were calculated using the data of the linear phase of the reaction. Natural log of (1-(cleavage ratio)) was plotted against the concentration of TEV protease, and the value of slope, k, was calculated as shown in TABLES 2-5.

TABLE 2
R49V (data plotted in FIG. 14A)

| Data Plotted | Control | R49V |
|---|---|---|
| Slope | y = −5.5178x − 0.0632 | y = −11.281x − 0.2037 |
| R² | 0.9409 | 0.968 |

| | |
|---|---|
| Formula | k_{R49V}/k_{control} = (k_{cat}/K_{M})_{R49V}/(k_{cat}/K_{M})_{control} |
| Values | 11.281/5.517 |
| Ratio | 2.0 |

TABLE 3
L76P (data plotted in FIG. 14B)

| Data Plotted | Control | L76P |
|---|---|---|
| Slope | y = −8.0486x − 0.0845 | y = −13.611x − 0.0596 |
| R² | 0.9842 | 0.9546 |

| | |
|---|---|
| Formula | k_{L76P}/k_{control} = (k_{cat}/K_{M})_{L76P}/(k_{cat}/K_{M})_{control} |
| Values | 13.611/8.0486 |
| Ratio | 1.7 |

TABLE 4
R49V_L76P (data plotted in FIG. 14C)

| Data Plotted | Control | R49V_L76P |
|---|---|---|
| Slope | y = −7.4133x − 0.0778 | y = −11.117x − 0.0916 |
| R² | 0.9791 | 0.9772 |

| | |
|---|---|
| Formula | k_{R49V}_{_{—}}_{L76P}/k_{control} = (k_{cat}/K_{M})_{R49V}_{_{—}}_{L76P}/(k_{cat}/K_{M})_{control} |
| Values | 11.117/7.413 |
| Ratio | 1.5 |

TABLE 5
R49F_L76P (data plotted in FIG. 14D)

| Data Plotted | Control | R49F_L76P |
|---|---|---|
| Slope | y = −5.2978x − 0.0231 | y = −13.518x − 0.0413 |
| R² | 0.9808 | 0.9973 |

| | |
|---|---|
| Formula | k_{R49F}_{_{—}}_{L76P}/k_{control} = (k_{cat}/K_{M})_{R49F}_{_{—}}_{L76P}/(k_{cat}/K_{M})_{control} |
| Values | 13.518/5.2978 |
| Ratio | 2.6 |

The calculated the differences in slope between the tested variant and the control are shown in FIGS. 14A-14D and indicate that the tested variant is more active than the control by that amount under the testing conditions. For example, the results shown in FIG. 14A and FIG. 14B respectively show that the R49V variant is 2.0 times more active and the L76P variant is 1.7 times more active than the control. Results shown in FIG. 14C indicate that the R49V_L76P variant is 1.5-times more active than the control TEV protease. As illustrated in FIG. 14D, the difference in slope between R49F_L76P variant and control TEV protease was 2.6-fold. This result indicates that R49F_L76P variant is 2.6-times more active than control TEV protease at 4° C.

### Example 14: Comparison to Commercial Enzyme

The activities of an R49F_L76P variant TEV protease produced in vivo in accordance with Example 1 and a commercially available TEV protease (New England Biolabs, Inc. Catalog #P8112S) on an example substrate, MBP-TEV-paramyosin ΔSal (SEQ ID NO:35), were compared at 4° C. Cleavage of the MBP-TEV-paramyosin ΔSal (70.7 kDa) at the TEV protease recognition site generates 2 products, MBP (43.7 kDa) and paramyosin ΔSal (27.3 kDa).

Activity assays were performed with 10 μg of MBP-TEV-paramyosin ΔSal mixed with 0.625 μg of TEVp in TEV Protease Reaction Buffer 1× in 50 μL final volume. A negative control (substrate+reaction buffer, no enzyme) and the two enzymatic reactions (each with 0.125 μg of enzyme per assay) were prepared on ice and then incubated at 4° C. for 5 hours. At commencement (T0), 30 minutes (T0.5), one hour (T1), three hours (T3), and five hours (T5), aliquots (10 μL each) were harvested, incubated at 65° C. for 10 minutes to inactivate the enzymes, and combined with 5 μL of 3×SDS Sample Buffer (New England Biolabs, Inc.). These volumes were then incubated at 95° C. for 2 minutes and spun at 14,000 rpm for 1 min. 10 μL of each was removed and loaded on Novex™ WedgeWell™ 4-20% Tris-Glycine gel (Invitrogen). After the gel was run, it was stained using SimplyBlue™ SafeStain (Life Technologies) and the bands of MBP-TEV-paramyosin ΔSal MBP, paramyosin ΔSal, and MBP were quantified using an Odyssey Imaging System (LI-COR Biosciences), from which the cleavage ratio of MBP-TEV-paramyosin ΔSal was calculated.

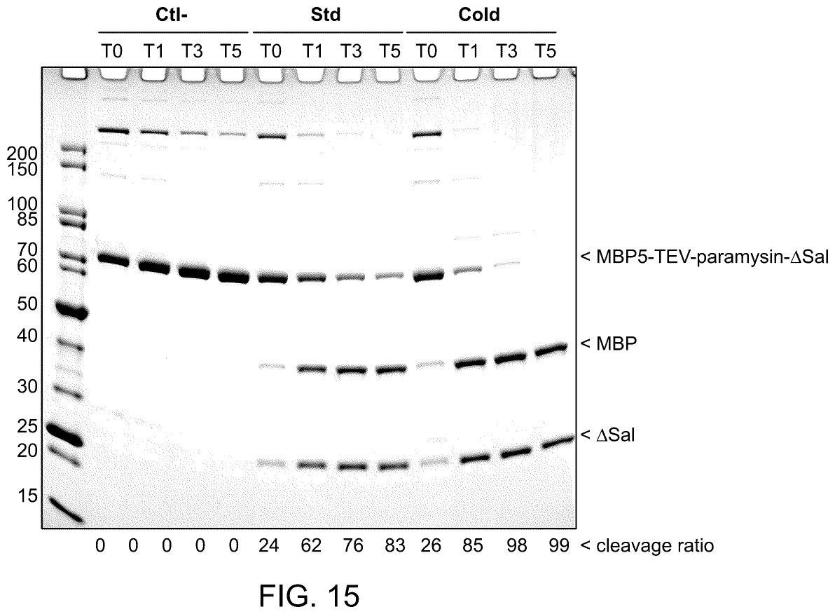

Results are shown in FIG. 15. Under the conditions tested, the variant protease outperformed the commercially available enzyme at every time point. The variant protease achieved 85% cleavage in just 1 hour at 4° C. while the commercially available enzyme only produced 62% cleavage in the same time. The variant TEV protease cleaved 98% of the substrate in 3 hours and 99% in 5 hours, while the commercial enzyme cleaved only 76% and 83% of the substrate at these times, respectively.

What is claimed is:

1. A cold-active variant TEV protease having an amino acid sequence at least 90% identical to SEQ ID NO:1, wherein X49 is any amino acid other than arginine, X73 is any amino acid other than glutamine, X76 is any amino acid other than leucine, X78 is any amino acid other than aspartate, X82 is any amino acid other than methionine, X83 is any amino acid other than isoleucine, X84 is any amino acid other than isoleucine, X89 is any amino acid other than lysine, X102 is any amino acid other than glutamate, X150 is any amino acid other than glutamine, X155 is any amino acid other than leucine, X181 is any amino acid other than serine, X184 is any amino acid other than lysine, X193 is any amino acid other than glutamine, X206 is any amino acid other than alanine, or X223 is any amino acid other than glutamate.

2. A cold-active variant TEV protease having an amino acid sequence at least 90% identical to SEQ ID NO:1, wherein at least one of X49, X73, X76, X78, X82, X83, X84, X89, X102, X150, X155, X181, X184, X193, X206, and X223 constitutes a substitution relative to the corresponding position of SEQ ID NO:31.

3. A cold-active variant TEV protease according to claim 1 having an amino acid sequence at least 98% identical to SEQ ID NO:2 or 3 or at least 97% identical to SEQ ID NO: 20 or 21.

4. A cold-active variant TEV protease according to claim 1, wherein (a) X49 is any amino acid other than arginine or (b) X76 is any amino acid other than leucine or (c) X49 is any amino acid other than arginine and X76 is any amino acid other than leucine.

5. A cold-active variant TEV protease according to claim 1, wherein (a) X49 is phenylalanine or valine or (b) X76 is proline or (c) X49 is phenylalanine or valine and X76 is proline.

6. A cold-active variant TEV protease according to claim 1, wherein the amino acid sequence comprises one or more of a substitution at position 45, a substitution at position 56, a substitution at position 135, and a substitution at position 219.

7. A cold-active variant TEV protease according to claim 1, wherein the amino acid sequence comprises one or more of a K45Q substitution, a K45R substitution, a L56V substitution, a S135G substitution, and a S219V substitution.

8. A cold-active variant TEV protease according to claim 1, wherein the amino acid sequence is identical to SEQ ID NO:1 at one or more of positions 2, 3, 12, 17, 23, 28, 30, 45, 50, 56, 58, 67, 68, 74, 77, 79, 80, 87, 90, 93, 106, 107, 110, 119, 120, 122, 127, 130, 132, 135, 138, 141, 146, 147, 148, 151, 153, 158, 162, 168, 170, 171, 173, 176, 177, 180, 203, 205, 209, 211, 215, 216, 217, 218, 219, 220, 224, 225, 226, 227, 228, 229, 235, 236, and 237.

9. A cold-active variant TEV protease comprising an amino acid sequence having:
(a) at least 90% identity to SEQ ID NO:1,
(b) at least one substitution relative to SEQ ID NO:31 at a position corresponding to position 49, 73, 76, 78, 82, 83, 84, 89, 102, 150, 155, 181, 184, 193, 206, 223 of SEQ ID NO:31,
(c) optionally a substitution at any of its positions or any combination of its positions corresponding to positions 56, 135, and 219 of SEQ ID NO:31, and
(d) optionally identity to wildtype TEV protease at any of its positions or any combination of its positions corresponding to positions 2, 3, 12, 17, 23, 28, 30, 45, 50, 56, 58, 67, 68, 74, 77, 79, 80, 87, 90, 93, 106, 107, 110, 119, 120, 122, 127, 130, 132, 135, 138, 141, 146, 147, 148, 151, 153, 158, 162, 168, 170, 171, 173, 176, 177, 180, 203, 205, 209, 211, 215, 216, 217, 218, 219, 220, 224, 225, 226, 227, 228, 229, 235, 236, 237 of SEQ ID NO:31.

10. A cold-active variant TEV protease comprising an amino acid sequence:
(a) having at least 90% identity to SEQ ID NO:1,
(b) having at least one substitution relative to wildtype TEV protease (SEQ ID NO: 31) at a position corresponding to position 49, 73, 76, 78, 82, 83, 84, 89, 102, 150, 155, 181, 184, 193, 206, 223 of SEQ ID NO:31,
(c) optionally having a substitution at any of its positions or any combination of its positions corresponding to positions 56, 135, and 219 of SEQ ID NO: 31,
(d) optionally lacking one or more of the following substitutions at its positions corresponding to: E2K, S3P, N12D, T17S, N23Q, H28L, H28Y, T30A, T30I, K45F, K45W, R50G, L56V, Q58F, Q58Y, Q58I, K67E, N68D, Q74L, I77V, G79E, R80S, M87L, M87T, D90G, P93S, E106G, E107D, C110S, K119E, S120R, S122P, D127A, C130S, F132L, F132S, S135G, S135F, I138T, K141R, T146A, T146C, T146S, K147E, D148R, D148P, D148A, C151A, S153C, S153N, T158A, F162S, F162A, S168T, S170A, N171D, N171Q, T173A, T173G, N176I, N176T, N177K, N177R, N177S, N177M, N177Y, T180A, R203Q, N205D, V209M, V209F, W211I, W211V, W211L, W211C, K215E, V216I, F217K, M218I, M218F, M218W, M218L, M218T, S219D, S219E, S219V, S219P, S219N, K220R, P224S, F225L, Q226stop, Q226S, Q226P, P227A, V228S, K229E, K229stop, M235K, N236S, and E237G of SEQ ID NO: 31; and
(e) optionally lacking one or more of the following substitutions at its positions corresponding to: K67P, M82I, 183V, Q150D, Q150H, S153L, L155A, L155M, and G213P of SEQ ID NO:31.

11. A cold-active variant TEV protease having an amino acid sequence at least 98% identical to SEQ ID NO:9 or 10 or at least 97% identical to SEQ ID NO:27 or 28 or at least 95% identical to SEQ ID NO:18 or 19.

12. A cold-active variant TEV protease according to claim 1, wherein the ratio of the k_{cat}/K_{M} of the variant TEV protease to the k_{cat}/K_{M} of a control TEV protease is in a range of 1.3-3.0.

13. A method comprising:
(a) contacting:
(i) a TEV protease substrate comprising a TEV protease recognition sequence having the amino acid sequence of SEQ ID NO:34, a protein of interest attached to the TEV protease recognition sequence, and a removable segment attached to the TEV protease recognition sequence, and
(ii) a variant TEV protease according to claim 1 to form a cleavage product mixture comprising separately the protein of interest and the removable segment; and
(b) optionally separating the protein of interest from the removable segment, wherein the contacting is performed at a temperature below 20° C., below 18° C., below 16° C., below 14° C., below 12° C., below 10° C., below 8° C., below 6° C., below 4° C., below 2° C. or below 0° C.

14. A method according to claim 13, wherein the ratio of the k_{cat}/K_{M} of the variant TEV protease to the k_{cat}/K_{M} of a control TEV protease is in a range of 1.3-3.0.

15. A method according to claim 13, wherein the variant TEV protease is a soluble variant TEV protease or an immobilized variant TEV protease.

16. A method comprising:
contacting:
(a) a composition comprising or potentially comprising a protein comprising a TEV protease recognition sequence having the amino acid sequence of SEQ ID NO:34, and
(b) a variant TEV protease according to claim 1 to produce a cleavage product mixture,
wherein the cleavage product mixture optionally
(i) is free of the intact protein comprising a TEV protease recognition sequence, and/or
(ii) comprises at least two fragments of the protein comprising the TEV protease recognition sequence, and, and
wherein the contacting is performed at a temperature below 20° C., below 18° C., below 16° C., below 14° C., below 12° C., below 10° C., below 8° C., below 6° C., below 4° C., below 2° C. or below 0° C.

17. A method according to claim 16, wherein the ratio of the k_{cat}/K_{M} of the variant TEV protease to the k_{cat}/K_{M} of a control TEV protease is in a range of 1.3-3.0.

18. A method according to claim 16, wherein the variant TEV protease is a soluble variant TEV protease or an immobilized variant TEV protease.

19. A method comprising:
(a) contacting:
(i) a TEV protease substrate comprising a TEV protease recognition sequence having the amino acid sequence of SEQ ID NO:34, a support attached to the TEV protease recognition sequence, and a bait molecule attached to the TEV protease recognition sequence, and
(ii) a composition comprising a target molecule capable of binding the bait molecule to form one or more TEV protease substrate: target molecule complexes, and
(b) contacting the one or more TEV protease substrate: target molecule complexes with a variant TEV protease according to claim 1 to cleave the TEV protease substrate at the recognition sequence,
wherein the (a) contacting is performed, the (b) contacting is performed, or the (a) contacting and the (b) contacting are performed a temperature below 20° C., below 18° C., below 16° C., below 14° C., below 12° C., below 10° C., below 8° C., below 6° C., below 4° C., below 2° C. or below 0° C.

20. A method according to claim 19, wherein the ratio of the k_{cat}/K_{M} of the variant TEV protease to the k_{cat}/K_{M} of a control TEV protease is in a range of 1.3-3.0.

21. A method according to claim 19, wherein the variant TEV protease is a soluble variant TEV protease or an immobilized variant TEV protease.

22. A kit comprising:
(a) a variant TEV protease according to claim 1; and
(b) a buffer,
wherein the variant TEV protease has a form selected from a liquid form, a gel form, an aqueous form, a film form, a crystalline form, a powder form, a cake form, a dried form, freeze dried form, a lyophilized form, and an immobilized form, and wherein the buffer is a reaction buffer or a storage buffer.
